(19) United States
(12) Patent Application Publication
(10) Pub. No.: US 2015/0359869 A1
(43) Pub. Date: Dec. 17, 2015

(54) METHODS AND COMPOSITIONS FOR PREVENTING A CONDITION

(71) Applicant: Cyvax, Inc., Atlanta, GA (US)

(72) Inventor: Richard MARKHAM, Columbia, MD (US)

(21) Appl. No.: 14/422,147

(22) PCT Filed: Aug. 14, 2013
(86) PCT No.: PCT/US2013/054985

§ 371 Date: Feb. 17, 2015

Related U.S. Application Data
(60) Provisional application No. 61/683,634, filed on Aug. 15, 2012.

(51) Int. Cl. A61K 39/015; A61K 9/00

(52) U.S. Cl. CPC A61K 39/015; A61K 9/0019; A61K 9/0009; A61K 2039/53

(57) ABSTRACT

Disclosed herein are nucleic acid-based vaccines against malaria and other conditions. A DNA construct comprising nucleic acid encoding one or more pathogen proteins, such as malaria parasite proteins, nucleic acid encoding a dendritic cell ligand, and a linker polynucleotide, is administered with an adjuvant and/or by electroporation to achieve in vivo results that are not achieved with the vaccine components alone. The vaccine can also be formulated using a fusion protein expressed by the disclosed nucleic acid, in combination with an adjuvant.

### CROSS-REFERENCE TO RELATED APPLICATION

This application claims the benefit of U.S. Provisional Application No. 61/683,634, filed Aug. 15, 2012 which is incorporated herein by reference in its entirety.

### BACKGROUND OF THE DISCLOSURE

For many pathogens, antigenic proteins are used as vaccines to raise a therapeutic and/or protective immune response in a human or animal. The proteins can be introduced in the form of nucleic acid encoding the protein. However, the ability of such vaccines to elicit sustained protective immune responses has been disappointing for some pathogens.

As one example, the malaria-causing Plasmodium (P.) parasite has proven particularly challenging as a vaccine candidate, in part because of its complex life cycle. The P. parasite is carried by mosquitoes and is introduced into a human when a mosquito bites the human injecting parasites along with anticoagulants. Sporozoite forms of the parasite invade liver hepatocytes, where the parasites are relatively shielded from the immune system and undergo asexual development during the next seven to ten days. During this time, the parasite number increases by 20,000 to 30,000-fold. The parasites are next released from the liver into the blood in the form of merozoites where they quickly enter red blood cells. The red blood cell stage of the disease is responsible for the main symptoms of malaria, including cyclic fevers. In the liver, an erythrocytic cycle becomes established with continued multiplication and liver cell release of merozoites. Some malaria parasites in the body differentiate into gametocytes, or sexual erythrocytic stages. These gametocytes can be ingested by biting mosquitos, leading to new sporogenic cycles and injection of the sporozoites into another human, thereby spreading the disease from human to human. Development of a malaria vaccine has also proven difficult because the P. parasite expresses different antigens at different stages of its life cycle. Four sets of antigens have been observed in the pre-liver; liver; blood and gametocyte stages.

### SUMMARY OF THE DISCLOSURE

The present disclosure provides vaccines that can be more effective than conventional vaccines. The disclosed vaccines combine an antigenic protein (antigen) with an immune cell targeting molecule. When the antigen and immune cell targeting molecule are co-administered, the immune cell's response to the antigen can be enhanced. The immune cell's response can be enhanced further by administering the vaccine with an adjuvant and/or through electroporation.

In certain embodiments, the vaccines can be provided as one or more proteins or polypeptides. In further embodiments, the vaccines can be provided as nucleic acid that encodes the antigen and immune cell targeting molecule. The nucleic acid vaccines can encode an antigen from a malaria parasite and an immune cell targeting molecule. The immune cell targeting molecule can target immature dendritic cells (iDC). The immune cell targeting molecule can target CXCR2, CXCR4, CCR1, CCR2, CCR3, CCR4, CCR5, CCR6, CCR7 and/or CCR8 receptors on iDC. In particular embodiments, the immune cell targeting molecule targets the CCR6 receptor on iDC. In further particular embodiments, the immune cell targeting molecule is macrophage inhibitory protein 3α (MIP-3α).

When the antigen is from a malaria parasite, particularly relevant antigens include those from P. falciparum, P. vivax, P. ovale or P. malariae parasites. In particular embodiments, the antigen is from P. falciparum. In particular embodiments, the antigen is the P. falciparum circumsporozoite protein (CSP) or a fragment or mimic thereof.

When an antigen is a CSP antigen, the antigen can comprise two or more NANP repeats, and the number of NANP repeats can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 60, 65, 70, 75, 80 or more. Each repeat can be the four amino acids NANP, NVDP, or NPDP. In additional embodiments, one of more of the NANP, NVDP or NPDP amino acids can be replaced with an amino acid that retains the characteristics of the original amino acid (i.e., a conserved substitution).

In any of the nucleic acid vaccines disclosed herein, the nucleic acid encoding a malarial antigen can be joined to the nucleic acid encoding the immune cell targeting molecule by a nucleic acid linker. In any of the vaccines disclosed herein, the linker nucleic acid can encode the peptide EFNDAQAPKS.

Any of the vaccines disclosed herein can be used in combination with an adjuvant. In certain embodiments, the adjuvant is or includes a cationic liposome. In other embodiments, the adjuvant comprises a commixture of GAP-DMORIE and DPyPE. In further embodiments, the adjuvant is Vaxfectin® (Vical Inc., San Diego, Calif.).

The present disclosure provides nucleic acid vaccines comprising DNA encoding P. falciparum CSP, linker DNA encoding the peptide EFNDAQAPKS, and DNA encoding the chemokine MIP-3α, wherein the vaccine is administered with the adjuvant Vaxfectin®. In particular embodiments, the nucleic acid vaccine comprising DNA encoding P. falciparum CSP, linker DNA encoding the peptide EFNDAQAPKS, and DNA encoding the chemokine MIP-3α is administered with the adjuvant Vaxfectin® and by electroporation.

Also provided are methods of enhancing immune response to an antigen or pathogen, comprising use of electroporation to enhance entry into cells of nucleic acid (in particular embodiments, DNA) encoding at least one pathogenic antigen or fragment or mimic thereof and at least one immune cell targeting molecule and, whereby the expressed antigen is taken up by iDC, thereby enhancing DC immune activity.

The present disclosure also provides methods of interrupting the malaria infection process in a human or other animal. Interrupting the infection process comprises preventing or reducing parasite entry into the liver, in particular embodiments by antibody-mediated neutralization of malaria sporozoites prior to liver entry. Antibodies capable of neutralizing malaria parasites prior to liver entry are specific for one or more epitopes of the malaria CSP.

In another aspect, methods for eliciting an immune response in a subject are provided comprising administering to the subject a pharmaceutical composition comprising a nucleic acid sequence encoding an antigen or fragment or mimic thereof from a parasite fused to an immune cell targeting molecule. In particular embodiments, the pharmaceutical composition further comprises an adjuvant. In particular embodiments, the immune response prevents or reduces the likelihood of the subject developing an infection from a parasite. In additional particular embodiments, the immune response treats an infection from a parasite in the subject. In more particular embodiments, the subject is a human. In additional particular embodiments, the subject is a non-human mammal.

### BRIEF DESCRIPTION OF THE DRAWINGS

FIG. 1 is a diagram illustrating that the adjuvant Vaxfectin® attracts dendritic and other inflammatory cells to sites of vaccination, and that MIP-3α specifically binds to the CCR6 receptor on iDC.

FIG. 2 is a diagram of three plasmid (p) constructs of malaria DNA vaccines and controls. M refers to MIP-3α; CSP refers to P. yoelii CSP; and MCSP refers to the fusion protein of MIP-3α and P. yoelii CSP. Shown are the positions of leader sequence, spacer, myc tag, and P. yoelii CSP amino acid sequence (SEQ ID NO:1).

FIG. 3 shows the expression of constructs of malaria DNA vaccines and controls. DNA vaccine candidate pMCSP and control constructs pCSP and pM were transfected into 293T cells. Expression of MIP-3α, CSP and MIP-3α-CSP fusion protein were detected with anti-myc tag antibody 48 hours after transfection.

FIG. 4 is a histogram showing results of an experiment in which C57BL/6 mice were immunized every two weeks for six weeks with 2 μg of the constructs of FIG. 3 with or without the Vaxfectin® formulation. For the positive control group (IrSpz), 10⁵ (initial immunization) and 5×10⁴ (booster immunizations) irradiated P. yoelii sporozoites (17XN) were inoculated at the same time-points. Mice were bled two weeks after the final immunization to determine specific antibody concentrations. Histograms represent means±the standard error of the mean (n=5) of antibody concentrations.

FIG. 5 is a histogram showing results of an experiment in which C57BL/6 mice were immunized with indicated DNA vaccine candidates and IrSpz as in FIG. 4. Two weeks after the final immunization, mice were challenged with 5×10³ P. yoelii sporozoites, and parasite-specific rRNA levels in the liver determined by quantitative RT-PCR. Results were expressed as mean±s.d. of copy numbers from 5 mice per group.

FIG. 6 is a histogram showing the results of a sporozoite neutralization assay. The assay was performed in a total volume of 100 μl that contained 1×10⁵ sporozoites in dissection medium and 10 μl of each immune serum from each immunized mouse. The mixture was incubated for 40 min on ice. The sporozoites were then added to HepG1.6 cell cultures that were maintained at 37° C. in 5% CO₂. At the end of 48 hours, cells were harvested. Total RNA was isolated and reverse transcription was performed. 18s rRNA were detected and quantified by real-time PCR.

FIG. 7 (top and bottom panels) shows results of experiments in which immunized mice were injected i.p. with anti-CD4, anti-CD8 or both mAbs for two days. 24 h after the last injection, the efficacy of the T-cell depletion was estimated by two-color flow cytometry analysis of peripheral blood lymphocytes, using FITC-conjugated anti-CD4 or APC-conjugated anti-CD8 mAbs (FIG. 7 top panel). The mice were then challenged with 2×10³ P. yoelii sporozoites, and parasite-specific rRNA levels in the liver determined by quantitative RT-PCR (FIG. 7 bottom panel). Results are expressed as mean±s.d. of copy numbers from 5 mice per group. Not shown in the FIG. is that all immunized mice received Vaxfectin® in addition to the indicated vaccines.

FIGS. 8A and 8B are histograms showing results of an experiment in which 2 μg of Vaxfectin®-formulated pMCSP and pCSP were inoculated into C57BL/6 mice tibialis anterior muscles, and then cytokine levels of injected muscle samples were analyzed by Real-time PCR 24 h after injection (FIG. 8A) and 48 h after injection (FIG. 8B).

FIG. 9 is a histogram showing the results of an experiment in which C57BL/6 mice were immunized with P. falciparum CSP (hMfCSP). Antibody response at 4 and 6 weeks was increasingly greater for the hMfCSP/Vaxfectin® composition than for the other groups indicated.

FIG. 10 is a histogram showing the results of an experiment in which C57/BL6 mice were challenged with 5000 sporozoites from transgenic P. berghei, a mouse-specific malaria parasite carrying a partial CSP protein from human P. falciparum, following vaccination with the indicated compositions. Plasmodium replication was expressed as 18s rRNA copy number. A significant reduction in replication was achieved only with the vaccination using hMfCSP and Vaxfectin® composition.

FIG. 11 is a histogram showing the results of an in vitro neutralization assay in HEPG2 cells using monkey 6160 serum and monkey 6166 serum at various dilutions. Results are expressed as P. berghei 18s rRNA copy numbers. Serum from monkeys 6160 and 6166 both showed neutralization at dilutions 1:6 and 1:12. The p value for the differences versus pre-immune sera is <0.01.

FIG. 12 is a bar graph showing the antibody response to P. yoelii CSP protein in C57BL/6 mice immunized with 2 μg of plasmid DNA encoding vaccine proteins and control proteins, as well as from control mice immunized with irradiated sporozoites (IrSpz). The DNA vaccines were administered with and without adjuvant Vaxfectin®, and the antibody responses compared with CSP DNA and MCSP DNA administered by electroporation.

FIG. 13 is a bar graph showing P. yoelii 18s rRNA copy numbers in livers of immunized mice from the experiment described in relation to FIG. 12, following challenge with 5000 sporozoites.

FIG. 14 shows the polynucleotide and amino acid sequence for a P. falciparum malaria vaccine, including CSP and human MIP-3α.

FIG. 15 provides sequences used in the hMfCSP vaccine construct (SEQ ID NO:2), including DNA encoding MIP-3α(SEQ ID NO:4) linked to DNA encoding a segment of the P. falciparum CSP (SEQ ID NO:6) via a spacer (SEQ ID NO:5); the DNA sequence also contains a human tissue plasminogen activator signal sequence (SEQ ID NO:3). Also shown are the corresponding polypeptide sequences (SEQ ID NO:7), and an alignment of the polynucleotide and polypeptide sequences (SEQ ID NO:8).

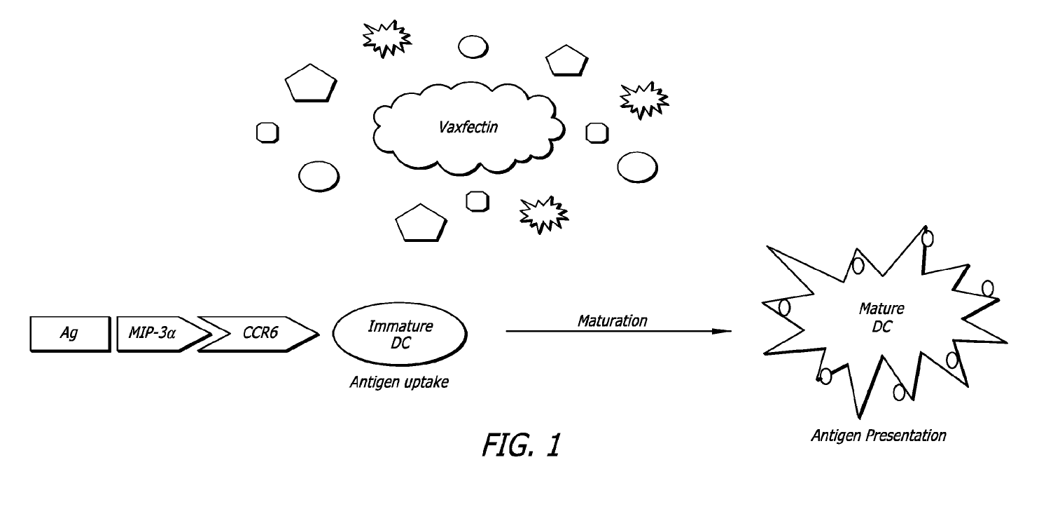

### DETAILED DESCRIPTION OF THE DISCLOSURE

The present disclosure provides vaccines that can be more effective than conventional vaccines. The disclosed vaccines provide nucleic acid sequences encoding an antigen and a molecule that targets an immune cell. When the antigen and molecule targeting an immune cell are co-expressed, the immune cell's response to the antigen can be enhanced. Without being bound by a specific mechanism, the vaccine acts with enhanced efficacy by creating a more effective interaction between the targeted immune cell and the antigen. The immune cell's response can be enhanced further by administering the nucleic acid sequence with an adjuvant and/or by delivering the vaccine using electroporation. Embodiments disclosed herein also include administration of the encoded proteins or polypeptides.

In particular embodiments disclosed herein, the targeted immune cell is a dendritic cell (DC), and more particularly, an immature dendritic cell (iDC). DCs (which include iDC) are also referred to as Langerhans cells when present in the skin (it is estimated that the human skin contains about 10⁹ Langerhans cells).

DCs are among the family of antigen-presenting cells in the immune system, and act as both initiators and modulators of the immune response to antigens from pathogenic and infectious agents. Antigen-presenting cells are those that ingest antigens and present aspects of the antigens on their surface to other cells, such as T cells and B cells, often then initiating further action by the other cells.

After activation by antigen, such as antigen of a vaccine disclosed herein, DCs migrate to the spleen, lymph node, and other lymphoid tissues where they mature and attract B cells through release of chemokines. B cells differentiate and produce antibody in response to DC stimulation. B cells can also be activated by direct exposure to antigen. DCs also play an important role in presenting antigen to T cells, including T helper cells, which are critical for an optimal B cell response to antigens. DCs also stimulate the production of antibodies through the secretion of soluble factors including the cytokine, IL-12. DCs express receptors for complement and Fc, thereby capturing and displaying antigen-antibody complexes assisting with the sustained proliferation of, and antibody production by, B cells.

After initial antigen ingestion (endocytosis) supporting the activities described above, DCs have reduced ability to intake additional antigen and will no longer react and initiate immune responses to newly-encountered antigens. The DC show, however, increased expression of cell-surface receptors CD80, CD86, and CD40, that can act as coreceptors in T cell activation. Therefore, following a first exposure to an antigen, a migrated mature DC can continue to present the initially encountered foreign antigen to naïve T cells. A T cell can be clonally expanded to effector T cells for a primary immune response. Some T cells differentiate to memory T cells for a second immune response in the event the same or a similar antigen is encountered in the future.

Prior to exposure to a specific antigen, DCs exist as iDC. iDC can be generated from hemopoietic bone marrow progenitor cells and exist in peripheral tissues and in secondary lymph nodes. Before exposure to antigen, iDC show low levels of MHC Class I and II expression and low T cell activation potential. In contrast, iDC have well-developed endocytic function. iDC survey the environment for pathogens using pattern recognition receptors (PRRs), e.g., Toll-like receptors (TLRs; TLRs are single membrane spanning receptors that recognize structurally conserved regions on microbes). Once an iDC ingests an antigen, it can process the antigen and present fragments to other cells on its surface using MHC molecules.

Based on the foregoing, and according to the present disclosure, vaccines can be especially effective when molecules targeted to an immune cell and expressed with antigen target iDC. Here, it is important to note that during maturation from iDC to DC, the expression of receptors on the cell surface changes. For example, while iDC express chemokine receptors CXCR2, CXCR4, CCR1, CCR2, CCR3, CCR4, CCR5, CCR6, CCR7, and CCR8, mature DCs lose the expression of most of these receptors, and gain expression of CXCR4, CXCR5 with enhanced expression of CCR7. Accordingly, preferred but not limiting embodiments disclosed herein can include chemokines directed to CXCR2, CXCR4, CCR1, CCR2, CCR3, CCR4, CCR5, CCR6, CCR7, and CCR8 receptors on iDC as immune targeting molecules. More particular embodiments include chemokines directed to CCR6 on iDC.

Without being bound by a specific mechanism, and when considering nucleic acid vaccines in their DNA formulations disclosed herein, the data suggest that the vaccines function by first introducing nucleic acid encoding a molecule directed to an iDC and an antigen into cells at or near the vaccination site, including but not limited to muscle cells. These cells express the linked iDC-directed molecule and antigen into the extracellular environment, and the iDC-directed molecule binds to its corresponding receptor on iDC. The iDC-directed molecule and antigen are taken into the iDC, where the antigen is processed to begin the process of DC maturation and migration and antigen presentation to T cells and B cells.

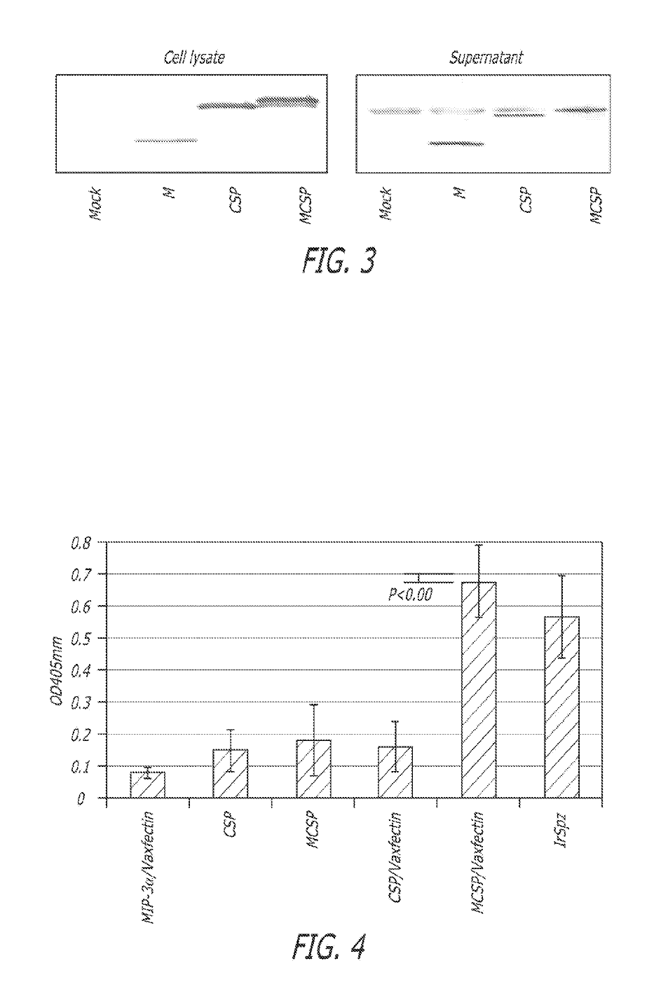

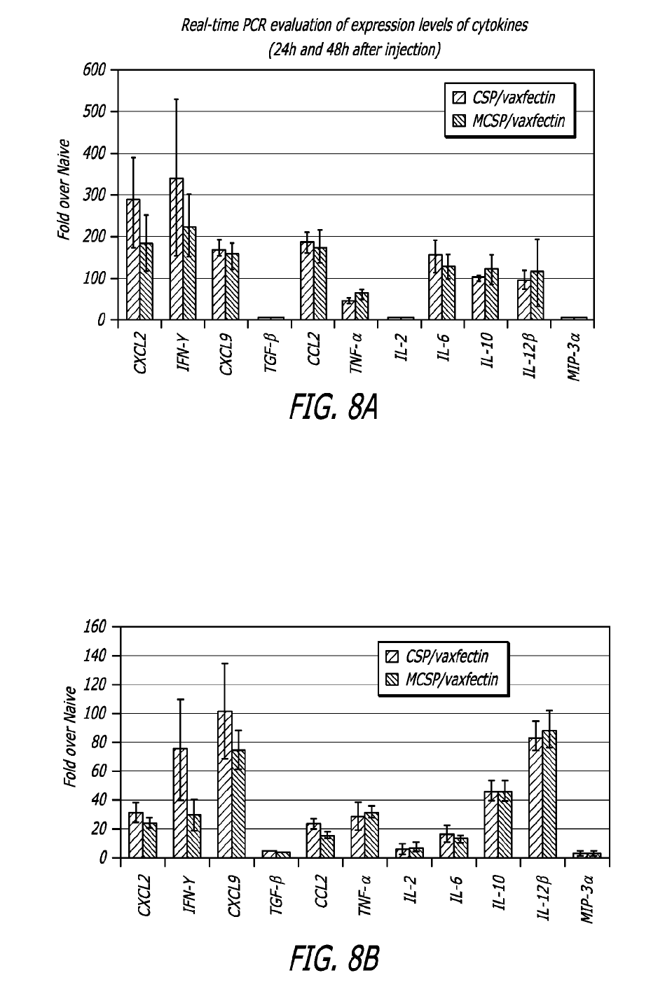

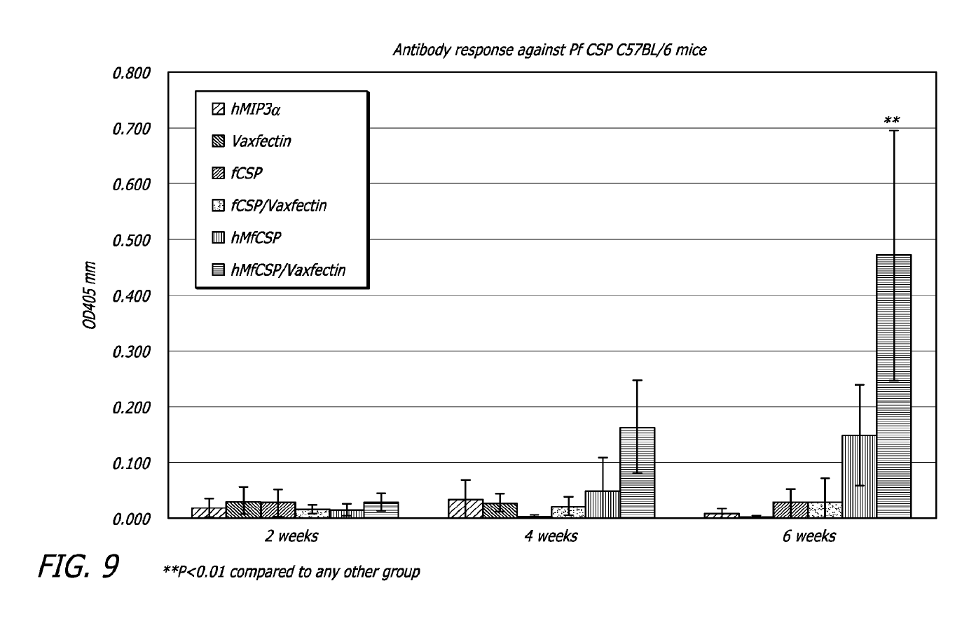

It should be noted that prior work on the immune response to malaria helped to identify DCs as antigen-presenting cells that prime antigen-specific CD4⁺ and CD8⁺ T cells. (Bruno-Romero et al., Infection and Immunity 69:5173-5176, 2001.) The DCs activate the appropriate T cells for effecting a response to malaria parasites. However, and without being bound by theory, the experiments described in Example 5 herein suggest that a DNA vaccine can achieve enhanced protective response to CSP without reliance on CD4⁺ and CD8⁺ T cell participation as the final effector cells. Particularly, and as shown in FIG. 3, a DNA vaccine encoding a MIP-3α-CSP fusion protein was expressed in 293T cells. Expression of MIP-3α did not change the expression of CSP. FIG. 8 shows that CD4+ and CD8+-T cell-depleted mice immunized with this DNA vaccine construct along with the adjuvant Vaxfectin® showed protection against subsequent malaria sporozoite challenge, and that this immune response was equivalent to the protection obtained using IrSpz. The data suggest that these T cells are not the final effector cells.

The next portion of this disclosure describes antigens and DC- or iDC-directed molecules appropriate for use with the vaccines of the present disclosure.

Parasite Antigens.

Antigens from the following parasites can be used in the vaccines disclosed herein: Acanthamoeba, African trypanosomiasis, Echinocococcus granulosus, Echinococcus multilocularis, Entamoeba histolytica, Trypanosoma cruzi, Angiostrongylus cantonensis, Babesia microti, Balantidium coli, Balamuthia mandrillaris, Baylisascaris, Schistosoma (S.) mansoni, S. haematobium, S. japonicum, S. masoni, S. intercalatum, Blastocystis hominis, Capillaria hepatica, Capillaria philippinensis, Austrobilharzia variglandis, Endolimax nana, Entamoeba coli, Entamoeba dispar, Entamoeba hartmanni, Entamoeba polecki, Iodamoeba buetschlii, Clonorchis sinensis, Ancylostoma (A.) brazilense, A. caninum, A. ceylanicum, Uncinaria stenocephala, Cryptosporidium, Cyclospora cayetanensis, Taenia, Cystoisospora beffi, Dientamoeba fragilis, Diphyllobothrium latum, Dipylidium caninum, Dracunculus medinensis, Giardia intestinalis, Brugia malayi, Entamoeba histolytica, Enterobius vermicularis, Fasciola hepatica, Fasciola gigantica, Fasciolopsis buski, Toxoplasma gondii, Trichinella spiralis, Giardia lamblia, Giardia duodenalis, Gnathostoma spinigerum, Heterophyes heterophyes, Hymenolepis nana, Leishmania promastigotes, Loa loa, Brachiola algerae, B. connori, B. vesicularum, Encephalitozoon cuniculi, E. hellem, E. intestinalis, E. bieneusi Microsporidium (M.) ceylonensis, M. africanum, Nosema ocularum, Trachipleistophora (T.) hominis, T. anthropophthera, Vittaforma corneae, Naegleria fowleri, Toxocara canis, Toxocara cati, Onchocerca volvulus, Opisthorchis felineus, Paragonimus westermani, Pneumocystis jirovecii, Sappinia diploidea, Sappinia pedata, Trypanosoma brucei, Trichuris trichiura, Ascaris lumbricoides, Ancylostoma duodenale, Necator americanus, Strongyloides stercoralis, Strongyloides füllebomi, Capillaria philippinensis, Taenia saginata, Taenia solium, Taenia asiatica, Toxoplasma gondii, Trichinella, or Trichomonas vaginalis.

Malaria parasites with antigen appropriate for use in the vaccines disclosed herein include P. vivax, P. ovale, P. falciparum, P. malariae, P. yoelii, P. bubalis, P. juxtanucleare, P. circumflexum, P. relictum, P. vaughani, P. minasense, P. agamae, or P. dominicum; the simian parasite P. knowlesi, which has caused human malaria; P. simiovale, which has a CSP variant of P. vivax, and P. brasilianum.

Nucleic acid vaccines described herein can encode antigens of any malaria parasite, particularly those that infect humans. Thus, antigens from one or more of P. falciparum P. vivax, P. ovale, and P. malariae are particularly suitable. Of these, CSP antigens specific to each of these parasites are highly relevant. These four species constitute the major human P. species. These species are most commonly differentiated at the post-liver erythrocyte stage, when they manifest differences in morphology and modifications of the red blood cells. The differences are best observed at a later stage than the present vaccines target, thus further supporting the broad applicability of the vaccine to all human P. species and infections.

Ongoing studies of human malaria infection may identify other species or antigen variants, and the vaccines disclosed herein can accommodate such subsequent findings without varying from the underlying mechanisms described herein.

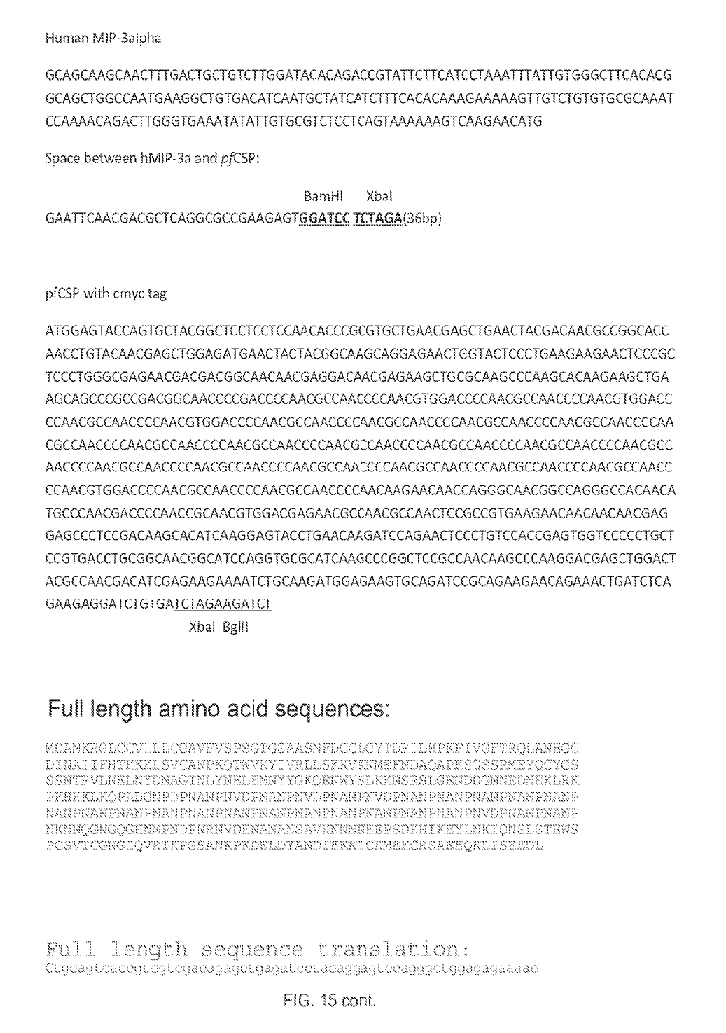

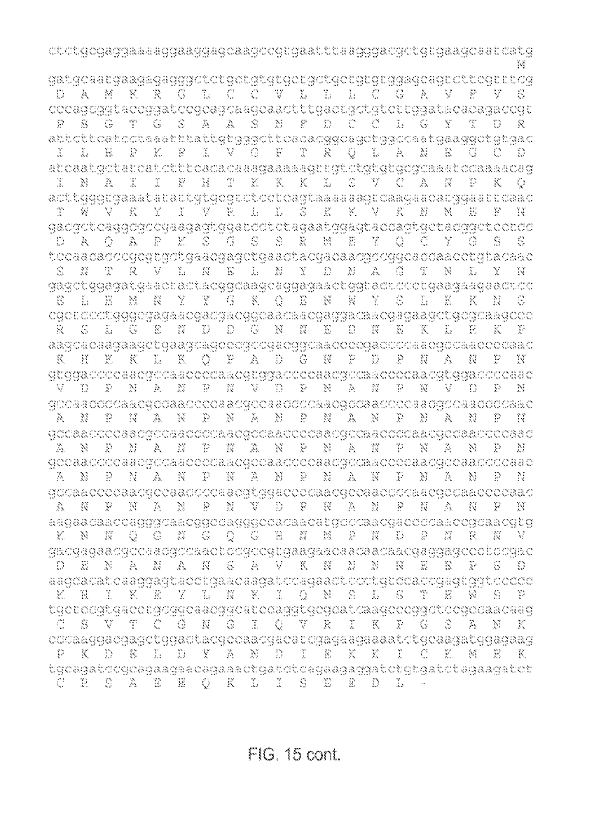

Fragments and/or mimics of the antigens described herein can also be used. For example, a fragment of the P. protein can be used. An exemplary amino acid sequence of P. falciparum is provided as SEQ ID NO: 6 (FIG. 15). The P. falciparum CSP protein has been extensively studied in isolates from infected humans, and a variety of epitope variants are publicly known and available, including specific fragments. Any length fragment of naturally occurring or synthetic CSP, up to and including full length, is suitable for the vaccines disclosed herein, as long as it is capable of processing by an iDC to yield an antigen portion that will elicit an immune response specific for the protein.

CSP proteins contain a region referred to as the “NANP” repeat region which includes a number of repeated four amino acid segments. Each one of these repeats consists of four amino acids, which can be asparagine (Asn, or N), alanine (Ala, or A), or proline (Pro, or P). One or more of these amino acids can be substituted with another and still be regarded as an NANP repeat. For example, as shown at amino acid positions 201-204 of SEQ ID NO:9, the second amino acid can be valine (position 202). As shown at amino acid positions 209-212 of SEQ ID NO:9, the third amino acid can be aspartic acid (Asp, or D). These two alternative positions can be found in one repeat, such as amino acid positions 217-220 of SEQ ID NO:9 (Asn Val Asp Pro, or NVDP). Other reported repeat sequences include NPDP (Asn Pro Asp Pro) as found at positions 193-196 of SEQ ID NO:9.

The NANP repeat region is an important component of CSP vaccines because it is targeted by antibodies of infected individuals. For the purposes of the vaccines disclosed herein, additional variations in amino acid sequence of the NANP repeat can be envisioned, as long as they maintain the underlying structure, enabling generation of an antibody response.

In addition to some flexibility in the NANP amino acid sequence, the coding region can be flexible, as long as the polynucleotide contains the appropriate codons for the amino acids of choice, as is known in the art. The NANP repeats need not all be contiguous, and individual repeats can be separated from each other by one or more amino acids. Again, the only limitation is the maintenance of the polypeptide structure for generating an antibody response against naturally-occurring malaria infection.

The number of NANP repeats in naturally occurring CSP varies from about 25 to at least 49 (Bowman et al., Science Reports 3:1990, 2013, which is incorporated by reference herein for its teaching regarding the same). In the vaccines disclosed herein, the number of NANP repeats can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 60, 65, 70, 75, 80 or more. Each repeat can be the four amino acids NANP, NVDP, or NPDP. Further, one or more of the NANP, NVDP or NPDP amino acids can be replaced with an amino acid that retains the characteristics of the original amino acid, referred to in the art as a conservative substitution.

In some embodiments, the antigen can be a MHC Class I epitope, a MHC Class II epitope, or a B cell epitope. A T cell epitope presented by MHC Class I molecules can be a peptide of approximately 8 to 11 amino acids. A T cell epitope presented by MHC Class II molecules can be longer than a MHC Class I molecule. A T cell epitope (MHC Class I or MHC Class II) web-based prediction tool is available from Immune Epitope Database Analysis Resource at, e.g.,

http://tools.immuneepitope.org/main/html/tcell_tools.html.

A web based B cell epitope prediction tool is available from Immune Epitope Database Analysis Resourceat, e.g.,

http://tools.immuneepitope.org/main/html/bcell_tools.html.

DC- or iDC-Directed Molecules.

Any molecule that targets a DC or iDC by, for example, binding to a receptor on the DC or iDC can be used if the interaction between the molecule and the DC or iDC triggers enhancement of DC or iDC activity. In this section, receptors on DC or iDC for targeting are described first and exemplary ligands are provided. Molecules known to target the receptors are described next. Following both of these descriptions, tables linking receptors to known ligands are provided.

DC or iDC Receptors to Target.

DCs can include myeloid DCs (mDC) and plasmacytoid DCs (pDC). mDCs express Toll-like receptors TLR-1, TLR-2, TLR-3, TLR-4, TLR-5, TLR-6, TLR-8, and/or TLR-11. Plasmacytoid DCs express Toll-like receptors TLR 7 and TLR 9. TLRs and DCs are reviewed, e.g., in Liu, J. Cancer Molecules 2:213-215 (2006) which is incorporated by reference herein for its teachings regarding the same.

CCR1 Receptor Binding.

In certain embodiments, the immune cell targeting molecule can bind the CCR1 receptor, e.g., a CCR1 receptor of an iDC. CCR1 (also known as CKR1; CD191; CKR-1; HM145; CMKBR1; MIP1aR; SCYAR1) is a member of the β chemokine receptor family, which can be a seven transmembrane protein similar to G protein-coupled receptors. CCR1 receptor ligands include, without limitation, macrophage inflammatory protein 1α (MIP-1α), regulated on activation normal T expressed and secreted protein (CCL5), monocyte chemoattractant protein 3 (MCP-3), and myeloid progenitor inhibitory factor-1 (MPIF-1).

CCR2 Receptor Binding.

In certain embodiments, the immune cell targeting molecule can bind the CCR2 receptor, e.g., a CCR2 receptor of an iDC. The CCR2 gene (also known as CKR2; CCR2A; CCR2B; CD192; CKR2A; CKR2B; CMKBR2; MCP-1-R; CC-CKR-2; FLJ78302; MGC103828; MGC111760; MGC168006) encodes two isoforms of a receptor. CCR2 receptor ligands include, without limitation, monocyte chemoattractant protein-1.

CCR5 Receptor Binding.

In certain embodiments, the immune cell targeting molecule can bind the CCR5 receptor, e.g., a CCR5 receptor of an iDC. CCR5 (also known as CKR5; CD195; CKR-5; CCCKR5; CMKBR5; IDDM22; CC-CKR-5; FLJ78003) is a member of the β chemokine receptor family, which can be a seven transmembrane protein similar to G protein-coupled receptors. This protein is expressed by T cells and macrophages, and is known to be a co-receptor for macrophage-tropic virus, including HIV, to enter host cells. Defective alleles of the CCR5 gene have been associated with HIV infection resistance. CCR5 receptor ligands include, without limitation, monocyte chemoattractant protein 2 (MCP-2), MIP-1α, MIP-1β and regulated on activation normal T expressed and secreted protein (CCL5).

CCR6 Receptor Binding.

In certain embodiments, the immune cell targeting molecule can bind the CCR6 receptor, e.g., a CCR6 receptor of an iDC. CCR6 is also known as BN-1; DCR2; DRY6; CCR-6; CD196; CKRL3; GPR29; CKR-L3; CMKBR6; GPRCY4; STRL22; CC-CKR-6; and C-C CKR-6). CCR6 is a member of the 13 chemokine receptor family and is predicted to be a seven transmembrane protein, similar to G protein-coupled receptors. The CCR6 gene can be expressed by iDC and memory T cells. CCR6 receptor can play a role in B-lineage maturation and antigen-driven B-cell differentiation, and it can regulate the migration and recruitment of dendritic and T cells during inflammatory and immunological responses. CCR6 receptor ligands include, without limitation, Human β-defensin 2 (also known as DEFB4A, BD-2, SAP1, DEFB2, HBD-2, DEFB-2, BEFB102), an antimicrobial peptide involved in innate immunity against infection and MIP-3α and microbially derived MIP-3α ligands such as, without limitation, viral β-defensin.

CXCR1 Receptor Binding.

In certain embodiments, the immune cell targeting molecule can bind the CXCR1 receptor, e.g., a CXCR1 receptor of an iDC. CXCR1 receptor 1, also known as CD128; CD181; CKR-1; IL8R1; IL8RA; CMKAR1; IL8RBA; CDw128a; C-C-CKR-1 is a member of the G-protein-coupled receptor family. CXCR1 ligands include, without limitation, IL8.

Molecules that Target DC or iDC Receptors.

Particular examples of molecules that target DC or iDC receptors include, without limitation, cytokines and chemokines or fragments or mimics thereof.

A cytokine can be a small cell-signaling molecule (e.g., a protein or peptide) secreted by a cell of the immune system that can be used in intercellular communication. Cytokines can act at nano-picomolar concentrations to modulate the activities of cells and tissues. They can mediate interactions between cells and regulate extracellular processes. A cytokine can be, e.g., a lymphokine, interleukin, or a chemokine. A cytokine can be, e.g., a monokine, interferon (IFN), or a colony stimulating factor (CSF).

In particular embodiments, the cytokine is a lymphokine. A lymphokine can be a protein produced by a lymphocyte, a type of white blood cell, e.g., a T cell. Lymphokines can function to attract immune cells, such as macrophages or other lymphocytes, to a site of infection. Examples of lymphokines include, e.g., interleukins (e.g., IL-1α, IL-1β, IL-2, IL-3, IL-4, IL-5, IL-6 (BSF-2), IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33 or IL-35). Interleukins can be synthesized by helper CD4+T lymphocytes, monocytes, macrophages, and endothelial cells. A lymphokine can be a colony-stimulating factor (CSF). A CSF can be a secreted glycoprotein that can bind to a receptor on the surface of a hemopoietic stem cell. CSFs include CSF1, CSF2, and CSF3.

IL8.

In particular embodiments, the immune cell targeting molecule is IL8. IL8 is also known as NAF; GCP1; LECT; LUCT; NAP1; CXCL8; GCP-1; LYNAP; MDNCF; MONAP; NAP-1. IL8 is secreted by several cell types and can function as a chemoattractant.

In particular embodiments, the immune cell targeting molecule is a chemokine. In more particular embodiments, the immune cell targeting molecule is a fragment or mimic of a chemokine. Examples of chemokines are provided, e.g., in Amanda Proudfoot, European Journal of Dermatology 8:147-157 (1998) which is incorporated by reference herein for its teachings regarding the same. A chemokine can induce chemotaxis in a nearby responsive cell. Chemokines can direct lymphocytes to lymph nodes.

Chemokines can be characterized as inflammatory (inducible) or homeostatic (constitutive), based on their pathophysiological activities. Inflammatory chemokines can be expressed during infection or tissue damage by resident and infiltrating leukocytes. In contrast, homeostatic chemokines can be produced constitutively in discrete microenvironments, and they can be involved in maintaining the physiological trafficking of immune cells.

Chemokines can be small proteins with a molecular mass of between about 8 to 10 kDa. One feature of many chemokines is four cysteines that form intramolecular disulphide bonds and affect the three dimensional shape of the chemokine. Chemokines can be classified into one of four different chemokine families based on the number and positioning of cysteines in the chemokine. A first family is the CC chemokine family. Members of the CC chemokine family have two adjacent cysteines near their amino terminus. CC chemokine family members include, e.g., CC chemokine ligands (CCL) 1 to 28, monocyte chemotactic proteins (MCP) 1, 2, and 3; macrophage inflammatory proteins (MIP) 1a and 18; and RANTES (CCL5). A second family is the CXC chemokine family. Members of the CXC chemokine family have two N-terminal cysteines separated by one amino acid. CXC chemokines include CXCL1-17, IL-8 and neutrophil-activating peptide-2 (NAP2, or CXCL7). A third family is the C chemokines. C chemokines lack adjacent cysteine residues. Examples of C chemokines include XCL1 and XCL2. A fourth family is the CXXXC, or CX₃C, family. CX3CL1 is an example of a member of the CX₃C family. Each of these can be used in the vaccines disclosed herein as DC or iDC targeting molecules.

In particular embodiments, the chemokine is CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXC12, CXCL13, CXCL14, CXCL15, CXCL16, CXCL17, XCL1, XCL2, or CX3CL1, or a fragment or mimic of any of these chemokines. The chemokine fragment or mimic thereof should retain the ability to bind to a chemokine receptor.

CCL2.

In particular embodiments, the immune cell targeting molecule is CCL2. CCL2 is also known as HC11; MCAF; MCP1; MCP-1; SCYA2; GDCF-2; SMC-CF; HSMCR30; MGC9434. CCL2 can bind to, without limitation, CCR2 and CCR4.

CCL3.

In particular embodiments, the immune cell targeting molecule is CCL3. CCL3 is also known as MIP1A; SCYA3; G0S19-1; LD78ALPHA; MIP-1α. CCL3 is an inducible cytokine. CCL3, also known as MIP-1α, plays a role in inflammatory responses through binding to the receptors CCR1, CCR4 and CCR5.

CCL4.

In particular embodiments, the immune cell targeting molecule is CCL4. CCL4 is also known as ACT2; G-26; LAG1; SCYA2; SCYA4; MIP1B1; AT744.1; MGC104418; MGC126025; MGC126026; MIP-113. CCL4, also known as MIP-113 has specificity for CCR5 receptors. It can be a chemoattractant for natural killer cells, monocytes and a variety of other immune cells.

CCL5.

In particular embodiments, the immune cell targeting molecule is CCL5. CCL5 is also known as SISd; SCYA5; RANTES; TCP228; D17S136E; MGC17164. CCL5 functions as a chemoattractant for blood monocytes, memory T helper cells and eosinophils. It causes the release of histamine from basophils and activates eosinophils. CCL5 can function as one of the natural ligands for CCR5.

CCL7.

In particular embodiments, the immune cell targeting molecule is CCL7. CCL7 is also known as FIC; MARC; MCP3; NC28; MCP-3; SCYA6; SCYA7; MGC138463; MGC138465. CCL7, also known as monocyte chemotactic protein 3, is a secreted chemokine that can attract macrophages during inflammation and metastasis.

CCL8.

In particular embodiments, the immune cell targeting molecule is CCL8. CCL8 is also known as CKb8; MIP3; Ckb-8; MIP-3; MPIF-1; SCYA23; Ckb-8-1; CK-β-8. CCL8 displays chemotactic activity for monocytes, lymphocytes, basophils and eosinophils. By recruiting leukocytes to sites of inflammation this cytokine can contribute to tumor-associated leukocyte infiltration.

CCL23.

In particular embodiments, the immune cell targeting molecule is CCL23. CCL23 is also known as CKb8; MIP3; Ckb-8; MIP-3; MPIF-1; SCYA23; Ckb-8-1; CK-β-8. CCL23 displays chemotactic activity on resting T lymphocytes and monocytes, lower activity on neutrophils and no activity on activated T lymphocytes. CCL23 is an agonist at CC chemokine receptor 1.

MIP-3α(CCL20).

In particular embodiments, the immune cell targeting molecule is MIP-3α. In additional particular embodiments, the immune cell targeting molecule is a fragment of MIP-3α. MIP-3α can be a ligand of CCR6 receptor. MIP-3α is also known as CCL20, Ckb4, LARC (liver activation regulated chemokine), ST38, or SCYA20). MIP-3α can be chemotactic for lymphocytes and can attract neutrophils.

CCL22.

In particular embodiments, the immune cell targeting molecule is CCL22. CCL22 is also known as MDC; ABCD-1; SCYA22; STCP-1; DC/B-CK; MGC34554; or A-152E5.1. MDC; ABCD-1; SCYA22; STCP-1; DC/B-CK; MGC34554; A-152E5.1 CCL22 displays chemotactic activity for monocytes, DCs, natural killer cells and for chronically activated T lymphocytes. It also displays a mild activity for primary activated T lymphocytes. CCL22 can bind to chemokine receptor CCR4. This chemokine can play a role in the trafficking of activated T lymphocytes to inflammatory sites and other aspects of activated T lymphocyte physiology.

CXCL2.

In particular embodiments, the immune cell targeting molecule is CXCL2. CXCL2 is also known as GRO2; GROb; MIP2; MIP2A; SCYB2; MGSA-b; MIP-2a; or CINC-2a.

Molecules other than chemokines can also bind to chemokine receptors on DC and iDC. For example, the CCR6 receptor also interacts with human β-defensins, as demonstrated by the ability of β-defensin to displace a chemokine ligand for CCR6. Suitable β-defensins include but are not limited to human β-defensin 1 and human β-defensin 2, which possess chemotactic activity towards iDC. Portions of these β-defensins that retain the ability to bind to an iDC receptor, including but not limited to CCR6, are also suitable for use in the disclosed nucleic acid-based vaccines. It is routine in the art to prepare a fragment or portion of a β-defensin protein and measure chemotactic activity towards iDC using, for example, methods described in Yang et al., Science 286:525-528, 1999. Other suitable defensins and defensin-like proteins are disclosed in Biragyn et al., Blood 100:1153-1159 (2002) and in Biragyn et al., J. Immunol. 167:6644-6653 (2001). Each of these references are incorporated by reference herein for their teachings regarding the same.

In particular embodiments, a nucleic acid sequence is provided comprising a sequence that encodes an antigen fused to a defensin. In particular embodiments, a nucleic acid sequence is provided comprising a sequence that encodes an antigen fused to human β-defensin 2. In more particular embodiments, the immune cell targeting molecule that can bind the CCR6 receptor is human β-defensin-2. In additional particular embodiments, the immune cell targeting molecule is a fragment of human β-defensin-2. DEFB4A is a cysteine-rich cationic low molecular weight antimicrobial peptide. It can be produced by epithelial cells and can exhibit potent antimicrobial activity against Gram-negative bacteria and Candida. DEFB4A can be produced following stimulation of epithelial cells by contacting microorganisms such as Pseudomonas aeruginosa or cytokines such as TNF-α and IL-1β.

In certain embodiments, any protein that can specifically compete with a CCR6 ligand, particularly MIP-3α or β-defensin 1 or 2, is suitable to achieve the goal of enhancing the iDC response to antigen. Where embodiments described herein specify MIP-3α, additional particular embodiments utilizing human β-defensin 1; human β-defensin 2; and other monocyte chemotactic proteins are expressly included. Table 1 provides exemplary DC receptors as well as associated ligands.

TABLE 1

| DC receptor | Ligand |
|---|---|
| CCR6 | MIP-3α; Human β-defensin 1; Human β-defensin |
| | 2; Monocyte chemotactic proteins |
| CCR1 | MIP-1α; MIP-5; CCL5; MCP-2; MCP-3; MPIF-1 |
| CCR2 | MCP-1; MCP-4 |
| CCR3 | Eotaxin, Eotaxin-2; and Eotaxin-3; CCL5; MCP-1; |
| | MCP-3; MCP-4; MEK; MIP-5; LPS-iCK |
| CCR4 | MIP-1α; CCL22 |
| CCR5 | MIP-1α; MIP-1β; CCL5; MCP-2 |
| CCR7 | CCL-19; CCL-21 |
| CCR8 | CCL-1 |
| CXCR1 | IL-8 |
| CXCR2 | CXCL1; Epithelial-derived neutrophil-activating |
| | peptide-78; Granulocyte chemotactic protein; |
| | Neutrophil-activating peptide 2; Lipopolysaccharide- |
| | induced CXC chemokine |
| CXCR3 | CXCL9; CXCL10; CXCL11 |

Provided in Table 2 are nucleic acid sequences or protein sequences for immune cell targeting molecules that can target DC.

TABLE 2

| Antigen label | Nucleic acid sequence or protein sequence |
|---|---|
| DEFB4A mRNA | agactcagct cctggtgaag ctcccagcca tcagccatga gggtcttgta tctcctcttc |
| [Homo sapiens] | tcgttcctct tcatattcct gatgcctctt ccaggtgttt ttggtggtat aggcgatcct |
| NM_004942.2 | gttacctgcc ttaagagtgg agccatatgt catccagtct tttgccctag aaggtataaa |
| | caaattggca cctgtggtct ccctggaaca aaatgctgca aaaagccatg aggaggccaa |
| | gaagctgctg tggctgatgc ggattcagaa agggctccct catcagagac gtgcgacatg |
| | taaaccaaat taaactatgg tgtccaaaga tacgca |
| DEFB4A protein | mrvlyllfsf lfiflmplpg vfggigdpvt clksgaichp vfcprrykqi gtcglpgtkc |
| [Homo sapiens] | ckkp |
| ACCESSION | |
| O15263 | |
| Homo sapiens β- | caaatccata gggagctctg ccttaccatt gggttcctaa ttaactgagt gagtgggtgt |
| defensin 3 mRNA, | gttctgcatg gtgagaggca ttggaatgat gcatcagaaa acatgtcata atgtcatcac |
| complete cds. | tgtaatatga caagaattgc agctgtggct ggaaccttta taaagtgacc aagcacacct |
| ACCESSION | tttcatccag tctcagcgtg gggtgaagcc tagcagctat gaggatccat tatcttctgt |
| AF301470.1 | ttgctttgct cttcctgttt ttggtgcctg ttccaggtca tggaggaatc ataaacacat |
| | tacagaaata ttattgcaga gtcagaggcg gccggtgtgc tgtgctcagc tgccttccaa |
| | aggaggaaca gatcggcaag tgctcgacgc gtggccgaaa atgctgccga agaaagaaat |
| | aaaaaccctg aaacatg |
| β-Defensin | mrihyllfal lflflvpvpg hggiintlqk yycrvrggrc avlsclpkee qigkcstrgr |
| 3 protein | kkccrrk |
| [Homo sapiens] | |
| ACCESSION | |
| AAG22030.1 | |
| CCL20 (MIP-3α) | agaatataac agcactccca aagaactggg tactcaacac tgagcagatc tgttctttga |
| mRNA Homo sapiens | gctaaaaacc atgtgctgta ccaagagttt gctcctggct gctttgatgt cagtgctgct |
| chemokine (C-C | actccacctc tgcggcgaat cagaagcagc aagcaacttt gactgctgtc ttggatacac |
| motif) ligand 2 | agaccgtatt cttcatccta aatttattgt gggcttcaca cggcagctgg ccaatgaagg |
| (CCL20), | ctgtgacatc aatgctatca tctttcacac aaagaaaaag ttgtctgtgt gcgcaaatcc |
| transcript | aaaacagact tgggtgaaat atattgtgcg tctcctcagt aaaaaagtca agaacatgta |
| variant 1 | aaaactgtgg cttttctgga atggaattgg acatagccca agaacagaaa gaaccttgct |
| Accession number: | ggggttggag gtttcacttg cacatcatgg agggtttagt gcttatctaa tttgtgcctc |
| NM_004591 | actggacttg tccaattaat gaagttgatt catattgcat catagtttgc tttgtttaag |
| | catcacatta aagttaaact gtattttatg ttatttatag ctgtaggttt tctgtgttta |
| | gctatttaat actaattttc cataagctat tttggtttag tgcaaagtat aaaattatat |
| | ttggggggga ataagattat atggactttc ttgcaagcaa caagctattt tttaaaaaaa |
| | actatttaac attcttttgt ttatattgtt ttgtctccta aattgttgta attgcattat |
| | aaaataagaa aaatattaat aagacaaata ttgaaaataa agaaacaaaa agttcttctg |
| | ttaaaaaaaa a |
| CCL20 protein | mcctksllla almsvlllhl cgeseaasnf dcclgytdri lhpkfivgft rqlanegcdi |
| human | naiifhtkkk lsvcanpkqt wvkyivrlls kkvknm |
| Accession number: | |
| P78556 | |
| Homo sapiens | agaatataac agcactccca aagaactggg tactcaacac tgagcagatc tgttctttga |
| chemokine (C-C | gctaaaaacc atgtgctgta ccaagagttt gctcctggct gctttgatgt cagtgctgct |
| motif) ligand 20 | actccacctc tgcggcgaat cagaagcaag caactttgac tgctgtcttg gatacacaga |
| (CCL20), | ccgtattctt catcctaaat ttattgtggg cttcacacgg cagctggcca atgaaggctg |
| transcript | tgacatcaat gctatcatct ttcacacaaa gaaaaagttg tctgtgtgcg caaatccaaa |
| variant 2, | acagacttgg gtgaaatata ttgtgcgtct cctcagtaaa aaagtcaaga acatgtaaaa |
| mRNA. | actgtggctt ttctggaatg gaattggaca tagcccaaga acagaaagaa ccttgctggg |
| ACCESSION | gttggaggtt tcacttgcac atcatggagg gtttagtgct tatctaattt gtgcctcact |
| NM_001130046 | ggacttgtcc aattaatgaa gttgattcat attgcatcat agtttgcttt gtttaagcat |
| | cacattaaag ttaaactgta ttttatgtta tttatagctg taggttttct gtgtttagct |
| | atttaatact aattttccat aagctatttt ggtttagtgc aaagtataaa attatatttg |
| | ggggggaata agattatatg gactttcttg caagcaacaa gctatttttt aaaaaaaact |
| | atttaacatt cttttgttta tattgttttg tctcctaaat tgttgtaatt gcattataaa |
| | ataagaaaaa tattaataag acaaatattg aaaataaaga aacaaaaagt tcttctgtta |
| | aaaaaaaa |
| Mus musculus | gagcactcgc agggcactgg gtacccagca ctgagtacat caactcctgg agctgagaat |
| chemokine (C-C | ggcctgcggt ggcaagcgtc tgctcttcct tgctttggca tgggtactgc tggctcacct |
| motif) ligand 20 | ctgcagccag gcagaagcag caagcaacta cgactgttgc ctctcgtaca tacagacgcc |
| (Ccl20),t | tcttccttcc agagctattg tgggtttcac aagacagatg gccgatgaag cttgtgacat |
| ranscript | taatgctatc atctttcaca cgaagaaaag aaaatctgtg tgcgctgatc caaagcagaa |
| variant 1, mRNA. | ctgggtgaaa agggctgtga acctcctcag cctaagagtc aagaagatgt aaaaaactga |
| ACCESSION | tgcttttttg ggatggaatt ggacacagcc caaggaggaa atgatcacag ctggggttga |
| NM_016960 | aggcttcacc tgcacatcac tgcacagacc tgatttgtgt cccagtggac ttgtccaatg |
| XM_484888 | gatgaagttg attcatattg catcatagtg tgtcatattt aagctcacat tagagttaag |
| | ttgtatttta tgttatttat agatctgaat tttctatgtt tagctattta atgttaattt |
| | cccacaatcc atgggggcgc ttagtggaag gattaatatt atgtttaagg gaatagttta |
| | tatggacctt tttgtcaaca ataagctatt gtaaagatat ttaatgttct gtttatttaa |
| | ttgcttctta aattgatatg attttcttat aaaacagaaa agaattataa gaatatattg |
| | aaaataaaag aattgaaagg taaaaaaaaa aaaaaaaaaa aaaaaaaaaa |
| | aaaaaaaaaa aaaaaaaaaa aa |
| Mus musculus | gagcactcgc agggcactgg gtacccagca ctgagtacat caactcctgg agctgagaat |
| chemokine (C-C | ggcctgcggt ggcaagcgtc tgctcttcct tgctttggca tgggtactgc tggctcacct |
| motif) ligand 20 | ctgcagccag gcagaagcaa gcaactacga ctgttgcctc tcgtacatac agacgcctct |
| (Ccl20), | tccttccaga gctattgtgg gtttcacaag acagatggcc gatgaagctt gtgacattaa |
| transcript | tgctatcatc tttcacacga agaaaagaaa atctgtgtgc gctgatccaa agcagaactg |
| variant 2, mRNA. | ggtgaaaagg gctgtgaacc tcctcagcct aagagtcaag aagatgtaaa aaactgatgc |
| ACCESSION | ttttttggga tggaattgga cacagcccaa ggaggaaatg atcacagctg gggttgaagg |
| NM_001159738 | cttcacctgc acatcactgc acagacctga tttgtgtccc agtggacttg tccaatggat |
| | gaagttgatt catattgcat catagtgtgt catatttaag ctcacattag agttaagttg |
| | tattttatgt tatttataga tctgaatttt ctatgtttag ctatttaatg ttaatttccc |
| | acaatccatg ggggcgctta gtggaaggat taatattatg tttaagggaa tagtttatat |
| | ggaccttttt gtcaacaata agctattgta aagatattta atgttctgtt tatttaattg |
| | cttcttaaat tgatatgatt ttcttataaa acagaaaaga attataagaa tatattgaaa |
| | ataaaagaat tgaaaggtaa aaaaaaaaaa aaaaaaaaaa aaaaaaaaaa |
| | aaaaaaaaaa |
| | aaaaaaaaa |
| CCL20 protein | macggkrllf lalawvllah lcsqaeaasn ydcclsyiqt plpsraivgf trqmadeacd |
| [Mus musculus] | inaiifhtkk rksvcadpkq nwvkravnll slrvkkm |
| Accession number: | |
| O89093 | |
| Homo sapiens | gctgcagagg attcctgcag aggatcaaga cagcacgtgg acctcgcaca gcctctccca |
| chemokine (C-C | caggtaccat gaaggtctcc gcggcagccc tcgctgtcat cctcattgct actgccctct |
| motif) ligand 5 | gcgctcctgc atctgcctcc ccatattcct cggacaccac accctgctgc tttgcctaca |
| (CCL5), mRNA | ttgcccgccc actgccccgt gcccacatca aggagtattt ctacaccagt ggcaagtgct |
| ACCESSION | ccaacccagc agtcgtcttt gtcacccgaa agaaccgcca agtgtgtgcc aacccagaga |
| NM_002985 | agaaatgggt tcgggagtac atcaactctt tggagatgag ctaggatgga gagtccttga |
| | acctgaactt acacaaattt gcctgtttct gcttgctctt gtcctagctt gggaggcttc |
| | ccctcactat cctaccccac ccgctccttg aagggcccag attctaccac acagcagcag |
| | ttacaaaaac cttccccagg ctggacgtgg tggctcacgc ctgtaatccc agcactttgg |
| | gaggccaagg tgggtggatc acttgaggtc aggagttcga gaccagcctg gccaacatga |
| | tgaaacccca tctctactaa aaatacaaaa aattagccgg gcgtggtagc gggcgcctgt |
| | agtcccagct actcgggagg ctgaggcagg agaatggcgt gaacccggga ggcggagctt |
| | gcagtgagcc gagatcgcgc cactgcactc cagcctgggc gacagagcga gactccgtct |
| | caaaaaaaaa aaaaaaaaaa aaaatacaaa aattagccgg gcgtggtggc ccacgcctgt |
| | aatcccagct actcgggagg ctaaggcagg aaaattgttt gaacccagga ggtggaggct |
| | gcagtgagct gagattgtgc cacttcactc cagcctgggt gacaaagtga gactccgtca |
| | caacaacaac aacaaaaagc ttccccaact aaagcctaga agagcttctg aggcgctgct |
| | ttgtcaaaag gaagtctcta ggttctgagc tctggctttg ccttggcttt gccagggctc |
| | tgtgaccagg aaggaagtca gcatgcctct agaggcaagg aggggaggaa cactgcactc |
| | ttaagcttcc gccgtctcaa cccclcacag gagcttactg gcaaacatga aaaatcggct |
| | taccattaaa gttctcaatg caaccataaa aaaaaaa |
| CCL5_HUMAN | mkvsaaalav iliatalcap asaspyssdt tpccfayiar plprahikey fytsgkcsnp |
| Protein | awfvtrknr qvcanpekkw vreyinslem s |
| [Homo sapiens] | |
| ACCESSION | |
| P13501 | |
| Mus musculus | cttgcagagg actctgagac agcacatgca tctcccacag cctctgccgc gggtaccatg |
| chemokine (C-C | aagatctctg cagctgccct caccatcatc ctcactgcag ccgccctctg cacccccgca |
| motif) ligand 5 | cctgcctcac catatggctc ggacaccact ccctgctgct ttgcctacct ctccctcgcg |
| (Ccl5), mRNA. | ctgcctcgtg cccacgtcaa ggagtatttc tacaccagca gcaagtgctc caatcttgca |
| ACCESSION | gtcgtgtttg tcactcgaag gaaccgccaa gtgtgtgcca acccagagaa gaagtgggtt |
| NM_013653 | caagaataca tcaactattt ggagatgagc taggatagag ggtttcttga ttctgaccct |
| | gtatagcttc cctgtcattg cttgctctag tcctagccag cttggggatg ccactcagta |
| | atcccctact cccactcggt cctgggaaaa tgggcatctc agctgctccg aggctctgca |
| | cagcaaaccc aagaaatcag catttcatta aaatttcaga tgcaaggaca aaaaaaaaaa |
| | aaaaaaaaaa aaaaaaaaaa aaaaaaaaaa aaaaaaaaa |
| Ccl5 | mkisaaalti iltaaalctp apaspygsdt tpccfaylsl alprahvkey fytsskcsnl |
| [Mus musculus] | avvfvtrmr qvcanpekkw vqeyinylem s |
| ACCESSION | |
| CAJ18523 | |
| Homo sapiens | agctggtttc agacttcaga aggacacggg cagcagacag tggtcagtcc tttcttggct |
| chemokine (C-C | ctgctgacac tcgagcccac attccgtcac ctgctcagaa tcatgcaggt ctccactgct |
| motif) ligand 3 | gcccttgctg tcctcctctg caccatggct ctctgcaacc agttctctgc atcacttgct |
| (CCL3), mRNA. | gctgacacgc cgaccgcctg ctgcttcagc tacacctccc ggcagattcc acagaatttc |
| ACCESSION | atagctgact actttgagac gagcagccag tgctccaagc ccggtgtcat cttcctaacc |
| NM_002983 | aagcgaagcc ggcaggtctg tgctgacccc agtgaggagt gggtccagaa atatgtcagc |
| | gacctggagc tgagtgcctg aggggtccag aagcttcgag gcccagcgac ctcggtgggc |
| | ccagtgggga ggagcaggag cctgagcctt gggaacatgc gtgtgacctc cacagctacc |
| | tcttctatgg actggttgtt gccaaacagc cacactgtgg gactcttctt aacttaaatt |
| | ttaatttatt tatactattt agtttttgta atttattttc gatttcacag tgtgtttgtg |
| | attgttlgct ctgagagttc ccctgtcccc tcccccttcc ctcacaccgc gtctggtgac |
| | aaccgagtgg ctgtcatcag cctgtgtagg cagtcatggc accaaagcca ccagactgac |
| | aaatgtgtat cggatgcttt tgttcagggc tgtgalcggc ctggggaaat aataaagatg |
| | ctcttttaaa aggtaaaaaa aaaaaaaaaa aaa |
| Chemokine (C-C | mqvstaalav llctmalcnq fsaslaadtp taccfsytsr qipqnfiady fetssqcskp |
| motif) ligand 3 | gvifltkrsr qvcadpseew vqkyvsdlel sa |
| [Homo sapiens]. | |
| ACCESSION | |
| AAH71834 | |
| Mus musculus | gggcatatgg cttcagacac cagaaggata caagcagcag cgagtaccag tcccttttct |
| chemokine (C-C | gttctgctga caagctcacc ctctgtcacc tgctcaacat catgaaggtc tccaccactg |
| motif) ligand 3 | cccttgctgt tcttctctgt accatgacac tctgcaacca agtcttctca gcgccatatg |
| (Ccl3), mRNA. | gagctgacac cccgactgcc tgctgcttct cctacagccg gaagattcca cgccaattca |
| ACCESSION | tcgttgacta ttttgaaacc agcagccttt gctcccagcc aggtgtcatt ttcctgacta |
| NM_011337 | agagaaaccg gcagatctgc gctgactcca aagagacctg ggtccaagaa tacatcactg |
| | acctggaact gaatgcctga gagtcttgga ggcagcgagg aaccccccaa acctccatgg |
| | gtcccgtgta gagcaggggc ttgagccccg gaacattcct gccacctgca tagctccatc |
| | tcctataagc tgtttgctgc caagtagcca catcgaggga ctcttcactt gaaattttat |
| | ttaatttaat cctattggtt taatactatt taattttgta atttatttta ttgtcatact |
| | tgtatttgtg actatttatt ctgaaagact tcaggacacg ttcctcaacc cccatctccc |
| | tcccagttgg tcacactgtt tggtgacagc tattctaggt agacatgatg acaaagtcat |
| | gaactgacaa atgtacaata gatgctttgt ttataccaga gaagtaataa atatgccctt |
| | taacaagtga aaaaaaaaaa aaaa |
| C-C motif | mkvsttalav llctmtlcnq vfsapygadt ptaccfsysr kiprqfivdy fetsslcsqp |
| chemokine 3 | gvifltkrnr qicadsketw vqeyitdlel na |
| [Mus musculus]. | |
| ACCESSION | |
| NP_035467 | |
| Mus musculus | ctctctggag tctgagtgcc ctttctacca gccatgagga ctctctgctc tctgctgctg |
| defensin β 2 | atatgctgcc tccttttctc atataccact ccagctgttg gaagtttaaa aagtattgga |
| (Defb2), mRNA | tacgaagcag aacttgacca ctgccacacc aatggagggt actgtgtcag agccatttgt |
| ACCESSION | cctccttctg ccaggcgtcc tgggagctgt ttcccagaga agaacccctg ttgcaagtac |
| NM_010030 | atgaaatgat tagaaggaag cacatggaag tcaagtgaca gatgtgtaat tgatgtttca |
| | ataaa |
| β-defensin 2 | mrtlcsllli ccllfsyttp avgslksigy eaeldhchtn ggycvraicp psarrpgscf |
| precursor [Mus | peknpcckym k |
| musculus | |
| ACCESSION | |
| NP_034160 | |

Two components of the vaccines disclosed herein, the antigen and immune cell targeting molecule, are discussed above. To further achieve an effective vaccine according to this disclosure, materials and methods are employed to enhance expression of the nucleic acid vaccine and/or to enhance availability of the vaccine for DC or iDC processing. One such method employs an adjuvant, and another employs administration through electroporation. Adjuvants and electroporation can be employed together. Both are discussed below.

Adjuvants.

The vaccines disclosed herein can be beneficially administered with adjuvants. The term “adjuvant” refers to material that modifies the effect of another agent in a vaccine. In particular instances, an adjuvant enhances the immune response to an antigen. In particular embodiments the pharmaceutical compositions described herein comprise or are administered with one or more different adjuvants (e.g., 1, 2, 3, 4, 5, 6, 7, 8, 9, 10 or more).

Adjuvants can function in a variety of ways. For example, an adjuvant can act as a releasing agent, presenting an antigen over a period of time (depot adjuvant). Traditionally, some depot adjuvants physically trap antigen at the site of injection, enhancing antigen presence at the site and slowing its release. This in turn prolongs and/or increases the recruitment and activation of antigen presenting cells, such as in this case DC or iDC. This mechanism played a role in a DNA vaccine tested by Schwendener et al., as reported in Methods Mol. Biol. 605:163-75 (2010). Intradermal injection of plasmid DNA liposomes encoding the gp33 glycoprotein of the lymphocytic choriomeningitis virus (LCMV) formed LCMV “antigen depots.” These antigen depots enabled long-lasting antigen loading of DCs in vivo, and was associated with a strong immune response. Without being bound by a specific mechanism, the use of liposome adjuvants in the present vaccine formulation may also facilitate sustained antigen loading of local DCs or iDC. This mechanism is consistent with the data presented in the Examples. Thus, adjuvants suitable for use herein include liposome formulations taught by Schwendener et al., Methods Mol. Biol. 605:163-75 (2010), which is incorporated by reference herein for its teaching regarding the same. A depot adjuvant can be, e.g., and oil emulsion.

Liposomes.

A pharmaceutical immunostimulatory agent can be encapsulated within a liposome using well-known technology. In particular embodiments, an adjuvant is a liposome. A liposome can be a particle comprising concentric lipid membranes containing phospholipids and other lipids in a bilayer configuration separated by aqueous compartments. Liposomes can be composed of naturally derived phospholipids or other surfactants. A liposome can encapsulate aqueous solution inside a hydrophobic membrane. Liposomes are described, e.g., in U.S. Pat. Nos. 6,586,409, 6,638,621, 6,989,195, 6,991,809, 7,105,229, 7,105,574, 7,537,768, 7,582,613, 7,628,993, and 7,655,235, which are incorporated by reference herein for their teachings regarding the same.

Liposomes can be a liposome from, e.g., Avanti Polar Lipids, Inc., Encapsula Nano Sciences (ENS), Taiwan Liposome Company (tlc), Liposome Company, Inc., Avestin, Inc, and Lyotropic Therapeutics. Liposome-based vaccines are described, e.g., in Schwender et al., Methods Mol Biol. 605: 163-175 (2010); Moon et al., Nature Materials 10: 243-251 (2011); Gregoriadis et al., Methods in Molecular Medicine vol. 29 pp. 305-311; Perrie et al., Vaccine vol. 19, pp. 3301-3310; Wang, Vaccine vol. 28 pp. 3134-42 (2010) which are all incorporated by reference herein for their teachings regarding the same.

Methods for preparing liposomes for administration to a subject are well known to those of skill in the art. U.S. Pat. No. 4,789,734, the contents of which are incorporated by reference herein for its teaching regarding the same, describes methods for encapsulating biological materials in liposomes. Essentially, the material can be dissolved in an aqueous solution, the appropriate phospholipids and lipids added, along with surfactants if required, and the material dialyzed or sonicated, as necessary. A review of known methods is also provided by G. Gregoriadis, Chapter 14, Drug Carriers in Biology and Medicine, pp. 2.sup. 87-341 (Academic Press, 1979) which is incorporated by reference herein for its teaching regarding the same.

In particular embodiments, the liposome comprises a cationic lipid. A cationic lipid can be an amphiphile that has a positive charge (at physiological pH) as measurable by instrumentation utilized at the time of the measurement. An amphiphile can be a molecule consisting of a water-soluble (hydrophilic) and an organic solvent-soluble (lipophilic) moiety. Where there are fatty acids or alkyl chains present on the cationic lipid, they can be 12-24 carbons in length, containing up to 6 unsaturations (double bonds), and linked to the backbone by either acyl or ether linkages; there can also only be one fatty acid or alkyl chain linked to the backbone. Where there is more than one fatty acid or alkyl chain linked to the backbone, the fatty acids can be different (asymmetric). Mixed formulations are also possible. In particular embodiments, the cationic lipid is GAP-DMORIE, DSTAP, DMTAP, DC-cholesterol, Ethyl PC, DDAB, dimethyldioctadecyl ammonium bromide; N-[1-(2,3-dioloyloxy)propyl]-N,N,N-trimethyl ammonium methylsulfate; 1,2-diacyloxy-3-trimethylammonium propanes, (including but not limited to, dioleoyl (DOTAP), dilauroyloxy, dimyristoyloxy, dipalmitoyloxy, and distearoyloxy); N-[1-(2,3-dioleoyloxy)propyl]-N,N-dimethyl amine; 1,2-diacyl-3-dimethylammonium propanes, (including but not limited to, dioleoyl (DODAP), dilauroyl. dimyristoyl, dipalmitoyl, and distearoyl); DOTMA, N-[1-[2,3-bis(oleyloxy)]propyl]-N,N,N-trimethylammonium chloride, (including but not limited to, dioleyl (DOTMA), dilauryl, dimyristyl, dipalmityl, and distearyl); DOGS, dioctadecylamidoglycylspermine; DC-cholesterol, 3β-[N—(N′,N′-dimethylaminoethane)carbamoyl]cholesterol; DOSPA, 2,3-dioleoyloxy-N-(2-(sperminecarboxamido)-ethyl)-N,N-dimethyl-1-propanam-inium trifluoro acetate; 1,2-diacyl-sn-glycero-3-ethylphosphocholines (including but not limited to dioleoyl (DOEPC), dilauroyl, dimyristoyl, dipalmitoyl, distearoyl, and palmitoyl-oleoyl); β-alanyl cholesterol; CTAB, cetyl trimethyl ammonium bromide; diC14-amidine, N-t-butyl-N′-tetradecyl-3-tetradecylaminopropionamidine; 14Dea2; TMAG, N-(α-trimethylammonioacetyl)didodecyl-D-glutamate chloride; O,O′-ditetradecanoyl-N-(trimethylammonioacetyl)diethanolamine chloride; DOSPER, 1,3-dioleoyloxy-2-(6-carboxy-spermyl)-propylamide; N,N,N′,N′-tetramethyl-N,N′-bis(2-hydroxylethyl)-2,3-dioleoyloxy-1,4-butan-ediammonium iodide; 1-[2-(acyloxy)ethyl]-2-alkyl (alkenyl)-3-(2-hydroxyethyl)imidazolinium chloride, derivatives as described by Solodin et al. (1995) Biochem. 43:13537-13544 which is incorporated by reference herein for its teaching regarding the same, such as DOTIM, 1-[2-(9(Z)-octadecenoyloxy)ethyl]-2-(8(Z)-heptadecenyl-3-(2-hydrox-yethyl) imidazolinium chloride; DPTIM, 1-[2-(hexadecanoyloxy)ethyl]-2-pentadecyl-3-(2-hydroxyethyl)imidazolinium chloride; 2,3-dialkyloxypropyl quaternary ammonium compound derivatives, contain a hydroxyalkyl moiety on the quaternary amine, as described in Feigner et al., (1994) J. Biol. Chem. 269:2550-2561 which is incorporated by reference herein for its teaching regarding the same, such as: DORI, 1,2-dioleoyl-3-dimethyl-hydroxyethyl ammonium bromide; DORIE, 1,2-dioleyloxypropyl-3-dimethyl-hydroxyethyl ammonium bromide; DORIE-HP, 1,2-dioleyloxypropyl-3-dimethyl-hydroxypropyl ammonium bromide; DORIE-HB, 1,2-dioleyloxypropyl-3-dimethyl-hydroxybutyl ammonium bromide; DORIE-HPe, 1,2-dioleyloxypropyl-3-dimethyl-hydroxypentyl ammonium bromide; DMRIE, 1,2-dimyristyloxypropyl-3-dimethyl-hydroxylethyl ammonium bromide; DPRIE, 1,2-dipalmityloxypropyl-3-dimethyl-hydroxyethyl ammonium bromide; or DSRIE, 1,2-disteryloxypropyl-3-dimethyl-hydroxyethyl ammonium bromide. Cationic lipids are described in U.S. Pat. No. 7,794,747, which is incorporated by reference herein for its teaching regarding the same.

In particular embodiments, the liposome comprises a neutral lipid, e.g., a neutral phospholipid. In more particular embodiments, the neutral phospholipid is DPyPE. In additional particular embodiments, a neutral lipid is, e.g., cholesterol; 1,2-diacyl-sn-glycero-3-phosphoethanolamines (including but not limited to dioleoyl (DOPE)); 1,2-diacyl-sn-glycero-3-phosphocholines; natural egg yolk or soy bean phosphatidylcholine (PC), and the like; or synthetic mono- and diacyl-phosphoethanolamines.

In particular embodiments, the liposome comprises a commixture of a cationic lipid and a neutral phospholipid which, when combined in an aqueous vehicle, self-assemble to form liposomes. In additional particular embodiments, the liposome comprises a commixture of (±)-N-(3-aminopropyl)-N,N-dimethyl-2,3-bis(cis-9-tetradecenyloxy)-1-propanaminium bromide (GAP-DMORIE) and 1,2-diphytanoyl-sn-glycero-3-phosphoethanolamine (DPyPE). In particular embodiments, the liposome that comprises a commixture of GAP-DMORIE and DPyPE is Vaxfectin®. Upon mixing with pharmaceutical immunostimulatory agents (e.g., nucleic acid sequence, protein, or vaccine disclosed herein), these cationic liposomes can associate through ionic, charge-based interactions with the pharmaceutical immunostimulatory agents and as a result provide an adjuvant effect, boosting the pharmaceutical immunostimulatory agent's (e.g., vaccine's) ability to stimulate immune responses. In mechanism of action studies, Vaxfectin® has been shown to increase a number of cytokines and chemokines, while Toll-like receptor signaling was contributory.

Vaxfectin® and its use are described in, for example, Hartikka et al., Vaccine 19:1911-1923 (2001) and Shlapobersky et al., Vaccine 2009 vol. 27: 6404-6410, which are incorporated by reference herein for their teachings regarding the same. Adjuvants with characteristics similar or the same as these Vaxfectin® characteristics may be used with embodiments disclosed herein.

An adjuvant can also be an irritant that amplifies an immune response. An adjuvant can also stabilize formulations of antigens. The precise mode of action is not understood for all adjuvants, but such lack of understanding does not prevent their clinical use for a wide variety of vaccines, whether protein-based or nucleic acid-based.

In particular embodiments, the adjuvant is a virosome. A virosome can comprise a unilamellar phospholipid bilayer vesicle that incorporates proteins derived from viruses that permit the virosome to fuse to target cells, e.g., cells of the immune system. A virosome can comprise a phospholipid bilayer membrane intercalated with viral envelope glycoproteins, e.g., influenza virus hemagglutinin (HA) and neuraminidase (NA). The HA and NA can confer structural stability and homogeneity to virosome particles. A virosome can be endocytosed by an antigen presenting cell, antigen synthesis/uptake can occur in the cell, the antigen can be proteolyzed, and the antigen can be presented on the cell for stimulation of T-cells. T-cell cytokines can stimulate B-cells to produce antibodies. Alternatively, if the antigen is displayed on the surface of the virosome, the antigen can directly stimulate B-cells to produce antibodies. A virosome can be provided by, e.g., Crucell, Pevion Biotech AG, or Virosome Biologicals B.V. In particular embodiments the virosome-based vaccines include but are not limited to, Epaxal or Inflexal.

In particular embodiments, the adjuvant is an inorganic adjuvant. For example, an adjuvant can be an aluminum salt. The aluminum salt can be, e.g., aluminum phosphate, aluminum hydroxide, aluminum potassium sulfate. An adjuvant can be calcium phosphate. Aluminum adjuvants can allow slow release of antigen.

In particular embodiments, the adjuvant comprises squalene. Squalene is an organic polymer termed a triterpene.

In particular embodiments, the adjuvant is an oil emulsion, product form bacteria, product from gram-negative bacteria, an endotoxin, cholesterol, fatty acids, aliphatic amines, or paraffinic or vegetable oil. In particular embodiments, the adjuvant can be the oil-in water emulsion MF59, ASO2, or ASO3. MF59 is a sub-micron oil-in-water emulsion of a squalene, polyoxyehtylene sorbitan monooleate (Tween 80) and sorbitan trioleate. The adjuvant can be ASO4 (aluminum and monophosphoryl lipid A).

In particular embodiments, the adjuvant is Freund's adjuvant. Freund's adjuvant comprises a water-in-oil emulsion of aqueous antigen in paraffin (mineral) oil of low specific gravity and low viscosity. Drakeol 6VR and Arlacel A (mannide monoleate) can be used as emulsifiers. Incomplete Freund's adjuvant comprises water-in-oil emulsion without added mycobacteria. Complete Freund's adjuvant comprises water-in-oil emulsion with heat-killed Mycobacterium tuberculosis or butyricum added.

In particular embodiments, microorganisms, or components of microorganisms, can be used as adjuvant including, e.g., Bordetella pertussis components, Corenybacterium derived P40 component, cholera toxin, and mycobacteria. In particular embodiments, the adjuvant is lipopolysaccharide (LPS).

In particular embodiments, the adjuvant includes CpG oligodeoxynucleotides (CpG ODN). CpGs are short single-stranded synthetic DNA molecules that contain a cytosine “C” followed by a guanine “G”. The “p” refers to the phosphodiester backbone of DNA, however some ODN can have a modified phosphorothioate (PS) backbone. When these CpG motifs are unmethlyated, they act as immunostimulants. CpG motifs are considered pathogen-associated molecular patterns (PAMPs) due to their abundance in microbial genomes but their rarity in vertebrate genomes. The CpG PAMP is recognized by the pattern recognition receptor (PRR) Toll-Like Receptor 9 (TLR9), which is expressed in B cells and plasmacytoid DCs (pDCs) in humans and other higher primates. Numerous sequences have been shown to stimulate TLR9 with variations in the number and location of CpG dimers, as well as the precise base sequences flanking the CpG dimers. This led to the creation of five classes or categories of CpG ODN based on their sequence, secondary structures, and effect on human peripheral blood mononuclear cells (PBMCs). The five classes are Class A (Type D), Class B (Type K), Class C, Class P, and Class S. The Class A ODNS have structural features that include: the presence of a poly G sequence at the 5′ end, the 3′ end, or both; an internal palindrome sequence; GC dinucleotides contained within the internal palindrome; and a partially PS-modified backbone. In particular embodiments the internal palindrome sequence can be 4 to 8 base pairs in length and vary in the order of bases. In more particular embodiments the palindrome sequence is 5′-Pu Pu CG Pu Py CG Py Py-3′. In additional particular embodiments Class A CpG ODNs can induce the production of large amounts of Type I interferons (e.g. IFNα) or induce the maturation of iDCs. The Class B ODNs have structural features that include: one or more 6mer CpG motif 5′-Pu Py C G Py Pu-3′; a fully phosphorothioated (PS-modified) backbone; and are generally 18 to 28 nucleotides in length and are strong stimulators of human B cell and monocyte maturation. In particular embodiments Class B ODNs stimulate human B cell and monocyte maturation. In particular embodiments Class B ODNs stimulate the maturation of pDCs or the production of small amounts of IFNα.

In particular embodiments, the adjuvant is an immunostimulating complex (ISCOM). An ISCOM can be a stable but non-covalently-bound complex of saponin adjuvant Quil-A, cholesterol, and amphipathic antigen in a molar ratio of approximately 1:1:1.

In particular embodiments, the adjuvant is a cytokine, e.g., any of the interleukins disclosed herein, hematopoietic factors such as granulocyte-macrophage colony stimulating factor (GM-CSF), granulocyte colony stimulating factor (G-CSF) and erythropoeitin, tumor necrosis factors (TNF) such as TNFα, lymphokines such as lymphotoxin, regulators of metabolic processes such as leptin, interferons such as interferon-α, interferon-δ, and interferon-γ, and/or any of the chemokines disclosed herein.

Electroporation for Tissue Delivery of Nucleic Acid Vaccines.

Nucleic acid immunization can be accomplished by electroporation for delivery of vaccine polynucleotides into cells. In particular embodiments, a pharmaceutical composition comprising a nucleic acid is administered by in vivo electroporation. In vivo electroporation can be performed with a syringe pre-loaded with nucleic acid sequence, and/or a pharmaceutical composition. The syringe and needle electrodes can be inserted into tissue, and the nucleic acid sequence, and/or pharmaceutical composition can be injected. A low micro-second electric pulse can be applied through the syringe needle. Electroporation can involve application of a millisecond electrical pulse, which can form an electric field. The electrical field can cause permeability in a cell membrane and can increase the uptake of biological material injected into local tissue. In vivo electroporation techniques are described in U.S. Patent Application Nos. 20090156787 and 20050052630, which are incorporated by reference herein for their teachings regarding the same. Electroporation devices are also described in U.S. Pat. Nos. 7,245,963, 6,912,417, 6,319,901, 6,278,895, 6,041,252, 5,873,849, 6,117,660 and 6,653,114, which are incorporated by reference herein for their teachings regarding the same. In vivo electroporation can be performed with technology from, without limitation, lnovio Pharmaceuticals, Inc., Ichor Medical Systems, or Cyto Pulse Sciences (Easy Vax Clinical Epidermal Electroporation System).

The electroporation administration methods described herein are not limited to a specific system or manufacturer, and one of ordinary skill in the art will be familiar with systems and apparati available. An exemplary method is an in vivo electroporation system, referred to as the Easy Vax, developed by Cyto Pulse, Baltimore, Md.; Harvard Apparatus, a division of Harvard Bioscience, Inc., Holliston, Mass.) This vaccine delivery system can deliver large molecules, using pulsed electric fields, directly in vivo into human skin cells to elicit an immune response against a specific target. The delivery system can include a single-use microneedle array in which each needle is coated with the polynucleotide of the vaccine. Hundreds of microneedles in the array can be aligned in 20 or more rows, with each row of needles dielectrically isolated. The array can be a few millimeters square and the needles can be <1 mm long.

Typically, when inserted into the skin, there are approximately 6200 epithelial cells and 25 Langerhans cells within the volume between any two rows when inserted 0.15 mm. The system can also include a Waveform Generator which can apply a pulsed voltage (1-50 volts) from one row of needles to the next. The electric field established between the needle rows can permeabilize the membranes of the cells between the rows permitting the polynucleotides to enter the cells.

This system includes several design features that enhance immunization. First, the electrode needles are only 150-500μ long, ensuring that the majority of the needles do not penetrate significantly beyond the basal lamina of the skin. Second, the needles can be spaced very close together, reducing the absolute voltage required to achieve cell membrane permeabilization. These design features can result in a painless delivery system and place the nucleic acid sequence at a site of abundant DCs/iDC to engage the proteins secreted by the cells that take up the nucleic acid.

The results of an experiment comparing immunization with vaccine DNA using the Cyto-Pulse Easy Vax system vs. immunization using the standard scarification technique with live vaccine demonstrated that equivalent ELISA and neutralization titers were obtained with either method. Additionally, previous studies indicate a dramatic enhancement of the response to DNA encoding HBsAg when electroporation using the Easy Vax system is added to the immunization regimen.

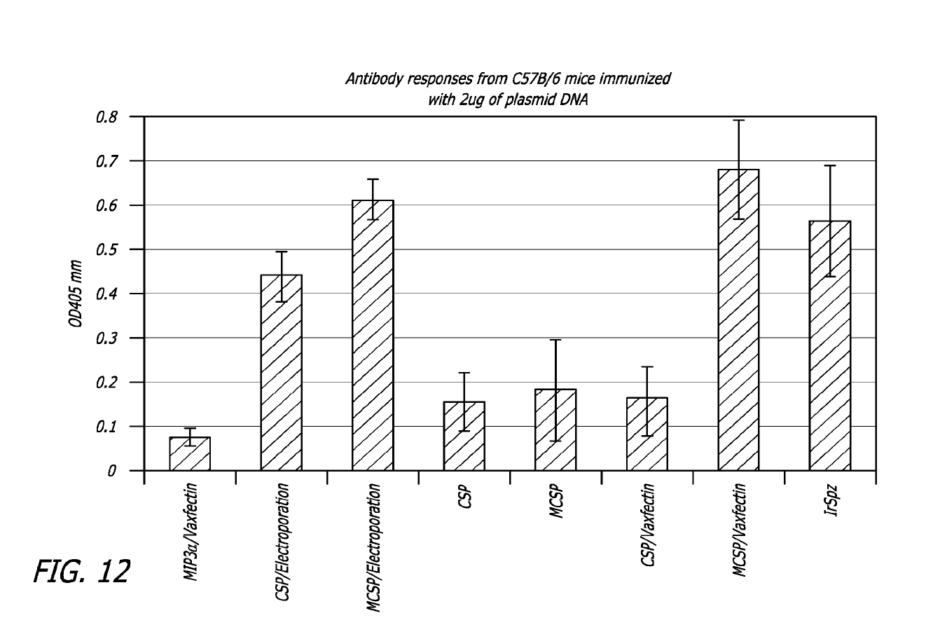

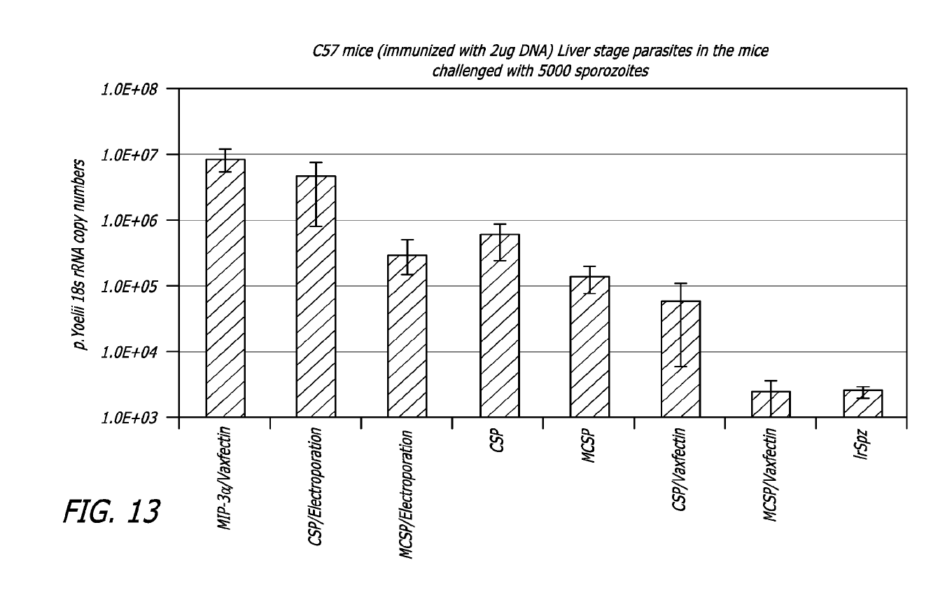

In the present disclosure, electroporation as described above is used with the nucleic acid vaccine constructs. As described above for the nucleic acid vaccine construct administered with adjuvant, the mechanism involves uptake of nucleic acid by cells at the site of administration, expression of the immune-cell targeting molecule/antigen, and association of the immune-cell targeting molecule with a corresponding receptor on DC or iDC in the vicinity of the vaccination. Data obtained from electroporation experiments are shown in Example 12 and in FIGS. 12 and 13. Without being bound by a specific mechanism, the data suggest that use of adjuvant brings more DCs (including iDC) to the site of inflammation, in this case due to vaccination. As a result, more iDC are available for binding to the antigen secreted by muscle cells, among other cells that have taken up the nucleic acid. Electroporation likely increases the uptake by the muscle cells and other cells in the vicinity of the vaccination, effectively making more antigen available for binding to iDC. Electroporation may also serve to bring more DCs (including iDC) to the site of inflammation. Both methods (adjuvant, electroporation) achieve the goal of increasing the efficiency of interaction between the vaccine protein and the available iDC: adjuvant achieves this by increasing the iDC numbers in the environment, and electroporation achieves this by increasing vaccine protein in the environment and/or increasing the iDC numbers in the environment. Combining electroporation with adjuvant use achieves the benefits of both mechanisms.

As previously stated, vaccines of the present disclosure are effective against malaria. These vaccines provide a breakthrough because the malaria-causing Plasmodium (P.) parasite has proven particularly challenging as a vaccine candidate (see the Background section). One malaria vaccine described herein is directed against sporozoites with the goal of preventing (or reducing) the parasites from entering and multiplying in the liver. Targeting sporozoites before liver entry also interferes with entry into the gametocyte stage, thereby reducing or eliminating human to human transfer of parasites.

The present disclosure is based on experiments described herein showing that a nucleic acid-based vaccine against a human pathogen can elicit an immune response equivalent to or better than those observed with antigens obtained from the pathogen. In particular embodiments, a nucleic acid-based vaccine against malaria elicited immune responses equivalent to or better than those observed with IrSpz. One aspect of the immune response generated by a vaccine described herein is the production of neutralizing antibodies against malaria CSP.

Particular vaccines disclosed herein were developed by expressing malaria antigens from nucleic acid constructs that fuse genes encoding malaria antigens to ligands for receptors on DCs and in some embodiments, iDC, and delivering the nucleic acid vaccine with an adjuvant such as the adjuvant Vaxfectin® or an adjuvant with similar characteristics and/or by delivering the vaccine using electroporation.

Particular embodiments disclosed herein include nucleic acid encoding an antigen fused to nucleic acid encoding an immune cell targeting molecule. In particular embodiments, the antigen is a malaria antigen. In particular embodiments the immune cell targeting molecule binds to a receptor on iDC. In more particular embodiments, the receptor on the iDC is one or more of: CXCR2, CXCR4, CCR1, CCR2, CCR3, CCR4, CCR5, CCR6, CCR7 or CCR8. In more particular embodiments, the antigen is a malaria antigen and the immune cell targeting molecule binds to a CXCR2, CXCR4, CCR1, CCR2, CCR3, CCR4, CCR5, CCR6, CCR7 or CCR8 receptor on iDC. In more particular embodiments, the antigen is a malaria antigen and the immune cell targeting molecule binds to a CCR6, receptor on iDC. In even more particular embodiments, the antigen is a malaria antigen and the immune cell targeting molecule is MIP3α.

In additional embodiments, the malaria antigen is derived from P. vivax, P. ovale, P. falciparum, P. malariae, P. yoelii, P. bubalis, P. juxtanucleare, P. circumflexum, P. relictum, P. vaughani, P. minasense, P. agamae, P. dominicum; P. knowlesi; P. simiovale, P. vivax, or P. brasilianum and the immune cell targeting molecule binds to a receptor on iDC. In particular embodiments, the malaria antigen is derived from P. vivax, P. ovale, P. falciparum, P. malariae, P. yoelii, P. bubalis, P. juxtanucleare, P. circumflexum, P. relictum, P. vaughani, P. minasense, P. agamae, P. dominicum; P. knowlesi; P. simiovale, P. vivax, or P. brasilianum and the immune cell targeting molecule binds to a CXCR2, CXCR4, CCR1, CCR2, CCR3, CCR4, CCR5, CCR6, CCR7 or CCR8 iDC receptor. In additional particular embodiments, the malaria antigen is derived from P. vivax, P. ovale, P. falciparum, P. malariae, P. yoelii, P. bubalis, P. juxtanucleare, P. circumflexum, P. relictum, P. vaughani, P. minasense, P. agamae, P. dominicum; P. knowlesi; P. simiovale, P. vivax, or P. brasilianum and the immune cell targeting molecule binds to a CCR6 iDC receptor. In particular embodiments, the malaria antigen is derived from P. vivax, P. ovale, P. falciparum, P. malariae, P. yoelii, P. bubalis, P. juxtanucleare, P. circumflexum, P. relictum, P. vaughani, P. minasense, P. agamae, P. dominicum; P. knowlesi; P. simiovale, P. vivax, or P. brasilianum and the immune cell targeting molecule is MIP3α.

In additional embodiments, the malaria antigen is derived from P. vivax, P. ovale, P. falciparum, P. malariae, and the immune cell targeting molecule binds to a receptor on iDC. In particular embodiments, the malaria antigen is derived from P. vivax, P. ovale, P. falciparum, P. malariae, and the immune cell targeting molecule binds to a CXCR2, CXCR4, CCR1, CCR2, CCR3, CCR4, CCR5, CCR6, CCR7 or CCR8 iDC receptor. In particular embodiments, the malaria antigen is derived from P. vivax, P. ovale, P. falciparum, P. malariae, and the immune cell targeting molecule binds to a CCR6 iDC receptor. In particular embodiments, the malaria antigen is derived from P. vivax, P. ovale, P. falciparum, P. malariae, and the immune cell targeting molecule is MIP3α. In particular embodiments, the malaria antigen is derived from P. falciparum and the immune cell targeting molecule is MIP3α.

In additional embodiments, the antigen is CSP or a fragment or mimic thereof derived from P. falciparum and the immune cell targeting molecule binds to a receptor on iDC. In particular embodiments, the antigen is CSP or a fragment or mimic thereof derived from P. falciparum, and the immune cell targeting molecule binds to a CXCR2, CXCR4, CCR1, CCR2, CCR3, CCR4, CCR5, CCR6, CCR7 or CCR8 iDC receptor. In particular embodiments, the antigen is CSP or a fragment or mimic thereof derived from P. falciparum, and the immune cell targeting molecule binds to a CCR6 iDC receptor. In particular embodiments, the antigen is CSP or a fragment or mimic thereof derived from P. falciparum, and the immune cell targeting molecule is MIP3α.

Each of the embodiments disclosed herein and particularly those described in preceding paragraphs [0108]-[0111] can be combined with an adjuvant. Each of the embodiments disclosed herein and particularly those described in preceding paragraphs [0108]-[0111] can be combined with a liposomal adjuvant. Each of the embodiments disclosed herein and particularly those described in preceding paragraphs [0108]-[0111] can be combined with a liposome comprising a cationic lipid and a neutral phospholipid. Each of the embodiments disclosed herein and particularly those described in preceding paragraphs [0108]-[0111] can be combined with a liposome comprising GAP-DMORIE and DPyPE. Each of the embodiments disclosed herein and particularly those described in preceding paragraphs [0108]-[0111] can be combined with Vaxfectin®.

The malaria antigen of each of the embodiments disclosed herein and particularly those described in preceding paragraphs [0108]-[0112] can include a NANP repeat region having 1-80 repeats. For example, SEQ ID NO:9 includes a NANP repeat region having 27 NANP repeats at positions 193-300. In further embodiments, 1, 2, 3, 4, 5, 6, 7, 8, 9, 10, 11, 12, 13, 14, 15, 16, 17, 18, 19, 20, 21, 22, 23, 24, or 25 of the NANP repeats at positions 193-300 are removed from a particular vaccine; one to 27 repeats is substituted by a repeat of a different amino acid sequence (NANP, NVDP, or NPDP, for example); and/or one or more NANP repeats is added before position 193, after position 300, or between any of the NANP repeats. In some embodiments, one or more amino acids, up to ten amino acids, can separate repeats.

In particular embodiments a pharmaceutical composition comprising a nucleic acid expressing an antigen (e.g., a malaria antigen) fused to an immune cell targeting molecule (e.g., MIP-3α) and an adjuvant (e.g., a liposome, such as a liposome comprising GAP-DMORIE and DPyPE) produces a synergistic immunological response when administered to a subject in need thereof. In particular embodiments the synergistic immunological response is directed to the antigen or a cell expressing the antigen. In particular embodiments a subject is administered a pharmaceutical composition comprising a nucleic acid expressing an antigen (e.g., a malaria antigen) fused to an immune cell targeting molecule (e.g., MIP-3α) and an adjuvant (e.g., a liposome, such as a liposome comprising GAP-DMORIE and DPyPE) produces a synergistic immunological response in the subject that results in a greater immunological response to the antigen. In particular embodiments the synergy prevents infection of the subject by a parasite comprising the antigen. In particular embodiments, administration of a pharmaceutical composition comprising a nucleic acid expressing an antigen (e.g., a malaria antigen) fused to an immune cell targeting molecule (e.g., MIP-3α) and an adjuvant (e.g., a liposome, such as a liposome comprising GAP-DMORIE and DPyPE) to a subject produces an immunological response that is greater than the addition of immunological responses observed from the administration to a subject of the adjuvant with nucleic acid sequence (e.g., DNA) encoding the antigen alone, or a fusion nucleic acid sequence (e.g., DNA) vaccine expressing a chemokine, but without the adjuvant.

In particular embodiments provided herein is a nucleic acid sequence encoding CSP or fragment or mimic thereof from P. falciparum fused to MIP-3α. In particular embodiments, provided herein is a pharmaceutical composition comprising a nucleic acid sequence encoding CSP or a fragment or mimic thereof from P. falciparum fused to MIP-3α, and Vaxfectin. The nucleic acid sequence encoding CSP and encoding MIP-3α can be separated by a spacer nucleic acid sequence. The protection against malaria provided by administration of this combination can be synergistic and exceed the sum of protection attained by using either the adjuvant with nucleic acid sequence (e.g., DNA) encoding the parasite antigen alone or a fusion nucleic acid sequence (e.g., DNA) vaccine with the chemokine, but without the adjuvant.

In particular embodiments, a malaria nucleic acid vaccine is provided comprising nucleic acid encoding a malaria antigen fused to nucleic acid encoding an immune cell targeting molecule that enhances immunological reactivity of the antigen. The nucleic acid fusion product can be administered with an adjuvant, e.g., a commercially available nucleic acid vaccine adjuvant. In particular embodiments, the combination of the antigen construct and the adjuvant can elicit a protective immune response in a mammal that is equivalent to, substantially similar to, or greater than the response elicited by IrSpz. In particular embodiments the mammal is a human. In additional particular embodiments the mammal is a non-human animal (e.g., a mouse, monkey, ape, dog, horse, cow, or deer).

By fusing the chemokine MIP-3α to the antigens of interest, iDC can be attracted to the site of antigen deposition and also ensure efficient uptake of antigen by the CCR6-bearing iDC that play a role in the initiation of immune responses. MIP-3α can attract iDC to dermal sites. GM-CSF can down-regulate expression of CCR6 on iDC, potentially interfering with their ability to initiate the optimal immune response. Interruption of CCR6 engagement can preclude the development of CD8+ T cell-mediated cytotoxic activity. By increasing the efficiency of both recruitment of iDC to the inoculation site and the uptake of antigen by those recruited iDC, the number of antigen-presenting cells that mature after antigen-uptake and migrate to sites of T cell activation can be expanded.

Nucleic Acid Sequence Properties.

In particular embodiments, the nucleic acid sequence is DNA, cDNA, RNA, mRNA, siRNA, miRNA, chromosomal DNA, genomic DNA, mitochondrial DNA, cell-free DNA, recombinant DNA, a plasmid (p), linear DNA, cosmid, shuttle p, virus, retrovirus, and/or artificial chromosome. In particular embodiments, a plasmid is provided comprising a sequence that encodes an antigen fused to an immune cell targeting molecule. In particular embodiments, a plasmid is provided comprising a sequence that encodes an antigen fused to a molecule that binds a DC. In particular embodiments, a plasmid is provided comprising a sequence that encodes an antigen fused to a ligand for a receptor on a DC. In particular embodiments, the plasmid can replicate in a mammalian cell. In particular embodiments the plasmid cannot replicate in a mammalian cell.

The plasmid can comprise a viral promoter. The promoter can be, e.g., SV40 enhancer and early promoter region or cytomegalovirus (CMV) immediate/early promoter.

The plasmid can comprise intron A, which can improve mRNA stability.

The plasmid can comprise a polyadenylation or transcriptional termination signal. For example, the polyadenylation signal can be the bovine growth hormone polyadenylation signal, rabbit β-globulin polyadenylation signal, or late SV40 polyadenylation signal.

In particular embodiments, the nucleic acid sequence is codon optimized for expression in a eukaryotic cell.

The plasmid can comprise an antibiotic resistance gene to facilitate replication of the plasmid in a microorganism, e.g., bacteria. The antibiotic resistance gene can permit growth of a microorganism harboring a plasmid with the antibiotic resistance gene in medium containing, e.g., ampicillin, kanamycin, or chloramphenicol.

In particular embodiments, the nucleic acid sequence comprises a leader sequence. In particular embodiments, the nucleic acid sequence comprises an N-terminal secretion sequence.

In particular embodiments, the nucleic acid sequence comprises a sequence between the sequence encoding the antigen and the sequence encoding the immune cell targeting molecule (i.e. spacer sequence). In particular embodiments, the spacer sequence is about 3 to 300 nucleotides, 3 to 240 nucleotides, 3 to 210 nucleotides, 3 to 180 nucleotides, 3 to 150 nucleotides, 3 to 120 nucleotides, 3 to 90 nucleotides, 3 to 60 nucleotides, or 3 to 36 nucleotides in length. In particular embodiments, the spacer sequence encodes the amino acid sequence: EFNDAQAPKSGS. In particular embodiments, the spacer sequence encodes an amino acid sequence that comprises at least about 10, 15, 20, 25, 30, 35, 40, 45, 50, 55, 60, 65, 70, 75, 80, 85, 90, or 95 percent serine, glycine, and/or alanine. In particular embodiments, the spacer sequence encodes at least 1, 2, or 3 prolines. In particular embodiments, the spacer sequence encodes at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35, 35 36, 37, 38, 39, or 40 amino acids. In particular embodiments, the spacer sequence encodes about 1-40, 1-30, 1-20, 1-15, 1-10, 1-5, 5-40, 5-30, 5-20, or 5-15 amino acids. In particular embodiments, the spacer sequence encodes the amino acid sequence (GGGS)₂GS, (GGGS)₃GS, (GGGS)₄GS, (GGGS)₅GS, or (GGGS)₆GS. In particular embodiments, the spacer sequence encodes the amino acid sequence GPGPG. The spacer sequence can allow for proper folding of the antigen and the immune cell targeting molecule, or the antigen and a molecule that binds a DC.

In particular embodiments, the nucleic acid sequence further expresses a T cell helper epitope. In particular embodiments, the T cell helper epitope is the pan DR epitope (PADRE).

The sequence encoding the antigen can be 5′ of the sequence encoding the immune cell targeting molecule. The sequence encoding the antigen can be 3′ of the sequence encoding the immune cell targeting molecule.

In particular embodiments, the nucleic acid sequence comprises sequence encoding an antigen fused to an immune cell targeting molecule, wherein the antigen fused to the immune cell targeting molecule is also fused to an epitope tag. In particular embodiments, the epitope tag is a Myc-tag, isopegtag, BCCP, calmodulin-tag, FLAG-tag, HA-tag, His-tag (e.g., 6His-tag), maltose binding protein-tag, Nus-tag, glutathione-S-transferase-(GST)-tag, green fluorescent protein-(GFP)-tag, thioredoxin-tag, S-tag, Softag-1, Softag 3, Strep-tag, SBP-tag, Ty tag, or V5-tag. The epitope tag can be a tandem tag. The epitope tag can comprise multiple copies of an epitope tag (e.g., 3×Myc-tag, 13×Myc-tag, 3×FLAG-tag, 3×HA-tag). The epitope tag can be used to evaluate protein secretion and to facilitate protein purification.

The sequence encoding the epitope tag can be 5′ of the sequence encoding the antigen. The sequence encoding the epitope tag can be 3′ of the sequence encoding the antigen. The antigen-immune cell targeting molecule protein, or an antigen fused to a molecule that binds a DC protein, expressed from a nucleic acid sequence, can have an epitope tag at the N-terminus, at the C-terminus, at the N-terminus and the C-terminus, internal, at the N-terminus and internal, at the C-terminus and internal, or at the N-terminus, internal, and at the C-terminus.

In particular embodiments, a plasmid is provided. In particular embodiments, DNA for a tissue plasminogen activator leader sequence and the DNA for MIP-3α (CCL20) fused to a codon optimized DNA, encoding portions of the P. falciparum CSP is inserted into pVR1012. In particular embodiments, a pVR1012 is synthesized to include restriction sites that permit insertion of sequences into the p.

The nucleic acid sequence can comprises one or more base changes (e.g., insertion, deletion, mutation) in an antigen sequence or immune cell targeting molecule sequence relative to a wild-type reference. The nucleic acid sequence can comprise one or more base changes in an antigen sequence or molecule that binds a DC. Changes to nucleic acid sequence can be made, e.g., with the QuikChange site-directed mutagenesis kit.

In certain embodiments, expression products (polypeptides) can be expressed by the nucleic acid sequences described herein and administered as a therapeutic composition.

The polypeptide can be synthesized in, for example, a bacteria, yeast, insect cell, or mammalian cell. The bacteria can be, e.g., E. coli. The E. coli strain can be BL21. The expression system can make use of a T7lac promoter. The polypeptide can be expressed from, e.g., a pET vector or a pBAD vector. The yeast can be, e.g., Saccharomyces cerevisiae or Pichia pastoris. The insect cell can be SF-9 or SF-21 ovarian cell lines from Spodoptera frugiperda, or High-Five cells (egg cells from Trichoplusia ni). A baculovirus can be used to express the polypeptide in an insect cell. The polypeptide can be produced by fermentation. The polypeptide can be synthesized in a cell free extract. A cell free expression system can couple transcription and translation. The cell free system can be, e.g., the Expressway™ Cell-Free Expression System from Invitrogen.

The polypeptide can be purified by conventional chromatography. Methods of purifying proteins are described, e.g., in Current Protocols in Protein Science, Print ISSN: 1934-3655, which is incorporated by reference herein for its teaching regarding the same.

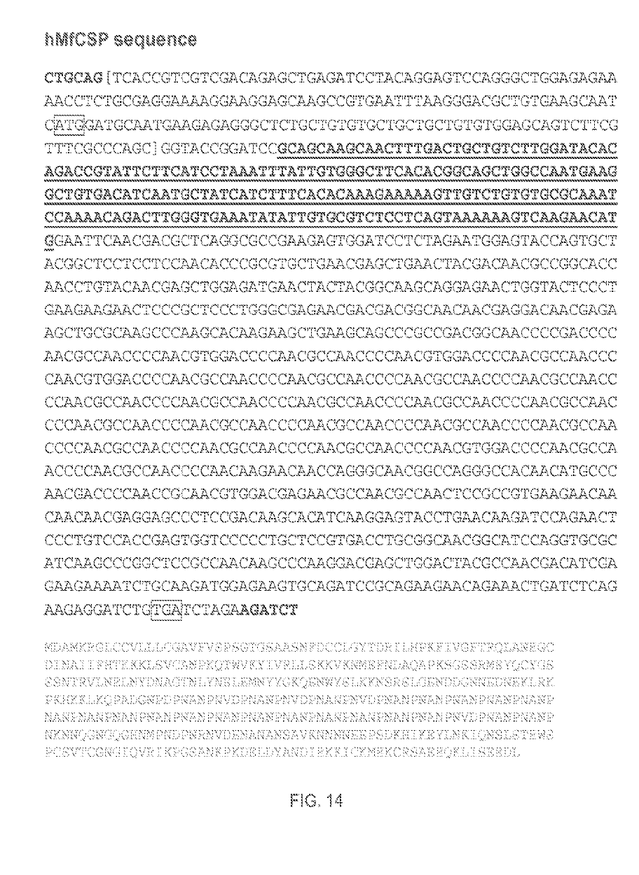

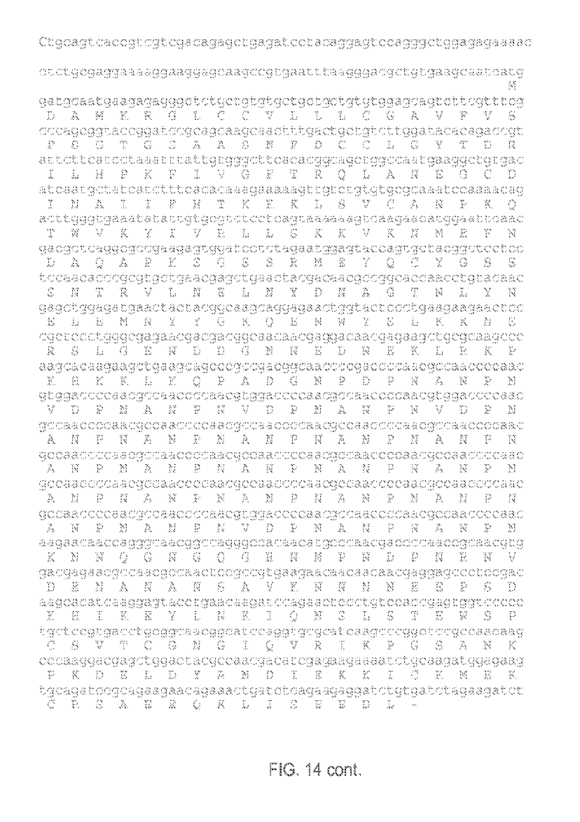

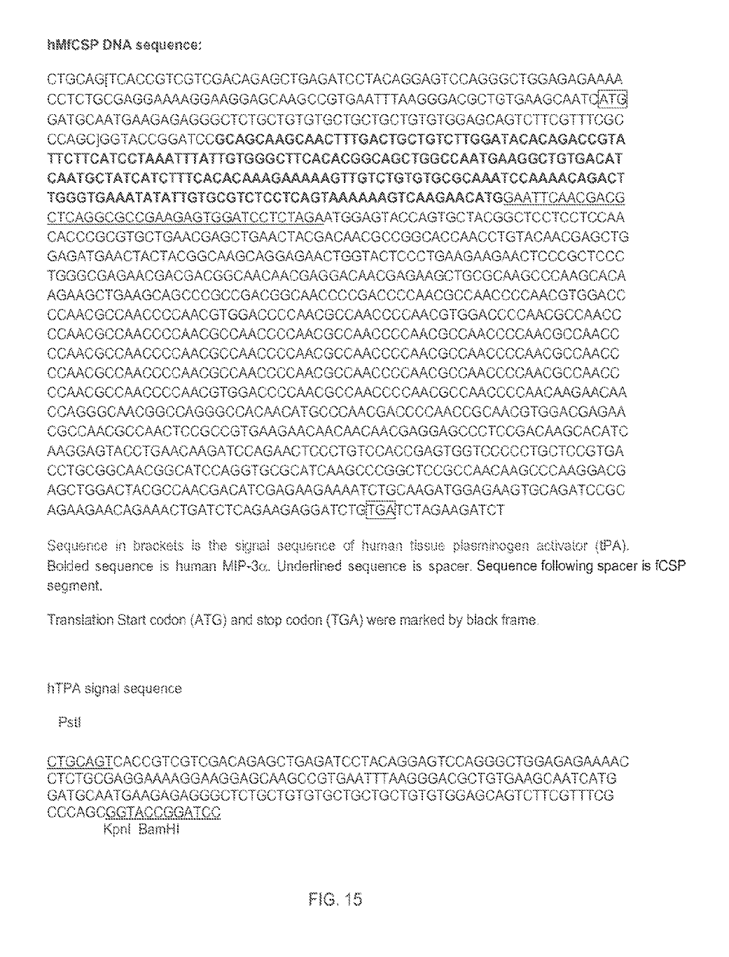

An exemplary plasmid for protein expression in shown in FIG. 14, which illustrates plasmid VR1012 into which was inserted the DNA for a tissue plasminogen activator leader sequence and the DNA for MIP-3α fused to a codon optimized DNA encoding portions of the P. falciparum CSP. Plasmid VR1012 is described in, for example, Hartikka et al., Hum. Gene Ther. 7: 1205-1217, 1996 which in incorporated by reference herein for its teachings regarding the same.

The term “fragment” or “protein fragment” can be a polypeptide that contains, for example between about 1 and 2000, 1 and 1950, 1 and 1900, 1 and 1850, 1 and 1800, 1 and 1750, 1 and 1700, 1 and 1650, 1 and 1600, 1 and 1550, 1 and 1500, 1 and 1450, 1 and 1400, 1 and 1350, 1 and 1300, 1 and 1250, 1 and 1200, 1 and 1150, 1 and 1100, 1 and 1050, 1 and 1000, 1 and 950, 1 and 900, 1 and 850, 1 and 800, 1 and 750, 1 and 700, 1 and 650, 1 and 600, 1 and 550, 1 and 500, 1 and 450, 1 and 400, 1 and 350, 1 and 300, 1 and 250, 1 and 200, 1 and 150, 1 and 100, or 1 and 50 contiguous amino acids, including all integers in between, of a reference polypeptide sequence. A fragment can be a polypeptide that contains, for example: about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, or more contiguous amino acids, including all integers in between, of a reference polypeptide sequence.

As used herein, a mimic has a sequence (amino acid or nucleic acid) that differs from a reference sequence but retains a substantially similar biological effect. A substantially similar biological effect is within 75% of a measured effect in a biological assay.

In particular embodiments, a polypeptide is provided comprising an antigen or a fragment or mimic thereof fused to an immune cell targeting molecule. In particular embodiments, a polypeptide is provided comprising an antigen or a fragment or mimic thereof fused to a molecule that binds a DC. The polypeptide can be any polypeptide that can be expressed from a nucleic acid sequence described herein. In particular embodiments the polypeptide is provided in a pharmaceutical composition for use in the treatment or prevention of a disease disclosed herein. In particular embodiments the polypeptide is a vaccine. Polypeptide vaccines are described in U.S. Patent Application Nos. 20090060915 and 20110027349, which are incorporated by reference herein for their teachings regarding the same.

Amino acids in a polypeptide can be in the L-isomeric form. The D-isomeric form of an amino acid can be substituted for the L-amino acid residue. NH₂ refers to the free amino group present at the amino terminus of a polypeptide, and COOH refers to the free carboxyl group present at the carboxyl terminus of a polypeptide. The amino acids herein can be represented by their standard 1-letter code or 3-letter code. An amino acid residue represented by “X” or “Xxx” refers to any one of the naturally occurring or non-naturally occurring amino acid residues known in the art or to a modification of a nearby residue. In keeping with standard protein nomenclature described in J. Biol. Chem., 1969, 247:3552-59, and adopted at 37 C.F.R. Sections 1.821-2461.822, all amino acid residue sequences represented herein by formulae have a left to right orientation in the conventional direction of amino-terminus to carboxyl-terminus. In addition, the phrase “amino acid residue” is broadly defined to include modified and unusual amino acids, such as those referred to in 37 C.F.R. Sections 1.821-1.822, and incorporated herein by reference. In a peptide or polypeptide, suitable conservative substitutions of amino acids are known to those of skill in this art and can be made generally without altering the biological activity of the resulting molecule. Watson et al., 1987, Molecular Biology of the Gene, 4th Edition, The Benjamin Cummings Pub. Co., p. 224), is incorporated herein by reference for its teachings regarding the same. Amino acid substitutions can be of single residues; such substitutions are preferably made with those set forth in Table 3, but can be of multiple residues, either clustered or dispersed. An amino acid can be replaced with a different naturally occurring or a non-conventional amino acid residue. Such substitutions can be classified as “conservative,” in which case an amino acid residue contained in a polypeptide is replaced with another naturally occurring amino acid of similar character either in relation to polarity, side chain functionality or size. Additions encompass the addition of one or more naturally occurring or non-conventional amino acid residues. Deletion encompasses the deletion of one or more amino acid residues.

TABLE 3
Conservative amino acid substitutions.

| Original residue | Conservative substitution(s) |
|---|---|
| Ala | Gly; Ser |
| Arg | Lys |
| Asn | Gln; His |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr, Ile |
| Phe | Met; Leu; Tyr |
| Ser | Thr |
| Thr | Ser |
| Trp | Tyr |
| Tyr | Trp; Phe |
| Val | Ile; Leu |

Substitutions can also be “non-conservative,” in which an amino acid residue which is present in a peptide is substituted with an amino acid having different properties, such as naturally-occurring amino acid from a different group (e.g., substituting a charged or hydrophobic amino acid with alanine), or alternatively, in which a naturally-occurring amino acid is substituted with a non-conventional amino acid.

The term “analog(s)” as used herein can refer to a composition that retains the same structure or function (e.g., binding to a receptor) as a polypeptide or nucleic acid sequence herein, such as the same gene from a different organism. Examples of analogs include mimetics or peptidomimetics, peptide, nucleic acids, small and large organic or inorganic compounds, as well as derivatives and variants of a polypeptide or nucleic acid herein. Such derivatives and variants refer to peptides and nucleic acids that differ from the naturally occurring polypeptides and nucleic acids by one or more amino acid or nucleic acid deletions, additions, substitutions or side-chain modifications. In some embodiments, a peptide analog is a peptide in which one or more of the amino acids has undergone side-chain modifications. Examples of side-chain modifications contemplated by the present disclosure include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄. In some embodiments, a peptide analog is one in which the guanidine group of arginine residue(s) is modified by the formation of heterocyclic condensation products with reagents such as 2,3-butanedione, phenylglyoxal and glyoxal; carboxyl group(s) is modified by carbodiimide activation via O-acylisourea formation followed by subsequent derivitisation, for example, to a corresponding amide; sulphydryl group(s) can be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH. In any of the analogs herein, any modification of cysteine residues can or can not affect the ability of the peptide to form disulphide bonds. In some embodiments, a peptide analog comprises tryptophan residue(s) that are modified by, for example, by oxidation with N-bromosuccinimide or alkylation of the indole ring with 2-hydroxy-5-nitrobenzyl bromide or sulphenyl halides; tyrosine residues altered by nitration with tetranitromethane to form a 3-nitrotyrosine derivative; imidazole ring(s) of a histidine residue modification accomplished by alkylation with iodoacetic acid derivatives or N-carbethoxylation with diethylpyrocarbonate; proline residue(s) modified by, for example, hydroxylation in the 4-position; glycosylation variants from a completely unglycosylated molecule to a modified glycosylated molecule; and altered glycosylation patterns as a result from expression of recombinant molecules in different host cells.

Formulations, Routes of Administration, and Effective Doses. In another aspect, formulations of a pharmaceutical composition, means of administration a pharmaceutical composition by different routes, and effective doses of a pharmaceutical composition are provided herein. In particular embodiments a pharmaceutical composition comprises a pharmaceutical immunostimulatory agents described herein, e.g., a nucleic acid sequence or polypeptide described herein. Such pharmaceutical compositions can be used to prevent, inhibit, reduce the severity of, or treat a condition (e.g., malaria, parasite infection, etc.) as described herein.

Pharmaceutical immunostimulatory agents (e.g., nucleic acid sequence, polypeptide) described herein can be administered as pharmaceutical compositions including those suitable for oral (including buccal and sub-lingual), rectal, nasal, topical, transdermal patch, pulmonary, vaginal, suppository, or parenteral (including intramuscular, intraarterial, intrathecal, intradermal, intraperitoneal, subcutaneous and intravenous) administration or in a form suitable for administration by aerosolization, inhalation or insufflation. General information on drug delivery systems can be found in Ansel et al., Pharmaceutical Dosage Forms and Drug Delivery Systems (Lippencott Williams & Wilkins, Baltimore Md. (1999) which is incorporated by reference herein for its teachings regarding the same. In particular embodiments, a pharmaceutical composition comprising a pharmaceutical immunostimulatory agent is provided by parenteral administration. In particular embodiments parenteral administration comprises injection. In additional embodiments the pharmaceutical compositions are provided through electroporation.

In particular embodiments, a pharmaceutical composition comprises carriers and/or excipients (including but not limited to buffers, carbohydrates, mannitol, proteins, polypeptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In particular embodiments, a pharmaceutical composition is substantially free of preservatives. In particular embodiments, a pharmaceutical composition can contain at least one preservative. General methodology on pharmaceutical dosage forms is found in Ansel et al., Pharmaceutical Dosage Forms and Drug Delivery Systems (Lippencott Williams & Wilkins, Baltimore Md. (1999)), which is incorporated by reference herein for its teachings regarding the same. While any suitable carrier known to those of ordinary skill in the art can be employed to administer the pharmaceutical composition, the type of carrier will vary depending on the mode of administration. Suitable formulations and additional carriers are described, e.g., in Remington “The Science and Practice of Pharmacy” (20^{th} Ed., Lippincott Williams & Wilkins, Baltimore Md.), which is incorporated by reference herein for its teachings regarding the same.

As is understood by one of ordinary skill in the art, the vaccines disclosed herein are administered to a subject, including a human subject, at sufficient dosage and frequency to treat or prevent a disease or condition as determined by a treating physician at the time of administration based on relevant considerations. Such administrations can be daily, weekly, monthly, yearly, once a decade etc or repeated in these time intervals for set numbers of administrations.

Methods of Treatment or Prevention.

In another aspect, methods of using pharmaceutical compositions described herein are provided. In particular embodiments, a method is provided to use pharmaceutical compositions to prevent, inhibit, reduce the severity of, or treat a condition of a subject. The term “subject” as used herein includes humans as well as other mammals including mouse, cow, horse, camel, gorilla, chimpanzee, rabbit, pig, dog, cat, rat, monkey, lemur, etc including patient forms of humans and these other exemplary animals

The term “treating” as used herein includes achieving a therapeutic benefit and/or a prophylactic benefit. By therapeutic benefit is meant eradication or amelioration of a condition. Also, a therapeutic benefit can be achieved with the eradication or amelioration of one or more of the physiological symptoms associated with the underlying condition such that an improvement is observed in the subject, notwithstanding the fact that the subject can still be afflicted with the underlying condition.

For embodiments where treatment of a subject is desired, a pharmaceutical composition disclosed herein can be administered to a which is incorporated by reference herein for its teachings regarding the same with a disease or condition, such as parasite infection, e.g., malaria, or to a subject reporting one or more of the physiological symptoms of a condition, even though a diagnosis of the condition may not have been made. Administration of a pharmaceutical composition disclosed herein can treat, reduce, lessen, shorten and/or otherwise ameliorate the disease or condition. In particular embodiments the pharmaceutical composition produces an immune response to an antigen sufficient to treat infection by a disease or condition comprising the antigen. In particular embodiments the pharmaceutical composition can modulate the immune system.

For embodiments where a prophylactic benefit is desired (e.g., prevention), a pharmaceutical composition of the disclosure can be administered to a subject at risk of developing a condition, such as parasite infection, e.g., malaria, or to a subject reporting one or more of the physiological symptoms of a condition, even though a diagnosis of the condition may not have been made. Administration can prevent the condition from developing, or it can reduce, lessen, shorten and/or otherwise ameliorate the disease or condition that develops. In particular embodiments the pharmaceutical composition produces an immune response to an antigen sufficient to prevent infection by a disease comprising the antigen or development of a condition comprising the antigen. In particular embodiments the pharmaceutical composition can modulate the immune system.

In particular embodiments, administering a pharmaceutical composition to a subject comprising a nucleic acid sequence encoding a malaria antigen fused to an immune cell targeting molecule, e.g., MIP-3α, and an adjuvant, e.g., a liposome comprising a commixture of GAP-DMORIE and DPyPE, results in a synergistic reduction in liver stage parasites in a mammal infected with a malaria parasite relative to the sum of the effects of administration of a pharmaceutical composition comprising an adjuvant with a nucleic acid sequence that encodes the antigen without the immune cell targeting molecule and a pharmaceutical composition comprising a nucleic acid encoding an antigen fused to an immune cell targeting molecule but without the adjuvant.

Non-Human Animal Models.

Exemplary non-human animals that can be used to study the nucleic acid sequences, polypeptides, and pharmaceutical compositions described herein can include mice, rats, guinea pigs, hamsters, sheep, pigs, and primates. Mouse models can be used to study malaria. In particular embodiments, the non-human animal is an immunocompromised mouse, e.g., an immunocompromised mouse transgenic for urokinase-type plasminogen activator (uPA), e.g., an immunocompromised mouse comprising a transgene that provides for liver-specific production of uPA (e.g., an Alb-uPA transgene, Heckel et al., Cell 62:447 (1990) which is incorporated by reference herein for its teachings regarding the same). Mice that can be used to study the nucleic acid sequences, polypeptides, and pharmaceutical compositions described herein include the strains C57B7/6, C.B-17, C3H, BALB/c, C57131/6, AKR, BA, B10, 129, etc. JAX® Mice strain NOD.Cg-Prkdc^{scid} Il2rg^{tm1Wjl}/SzJ, 005557, abbreviated NSG for NOD scid gamma, a NOD scid strain with a null mutation of the interleukin 2 receptor gamma (IL2rg) chain, can be used to study antimalarial drugs. Jimenez-Diaz et al., Antimicrob Agents Chemother. Vol. 53, pp. 4533-6 (2009). Other mouse models for studying malaria infection are described in Angulo-Barturen et al., PLoS One, vol. 3 e2252 (2008), and Mohmmed A. Biochem Biophys Res Commun. Vol. 309 pp. 506-11 (2003). Mouse models for studying malaria are also described, in U.S. Pat. No. 7,273,963. Each of these references is incorporated by reference herein for its teachings regarding the same.

The effective amount for use in subjects can be determined from animal models. For example, a dose for humans can be formulated to achieve circulating, liver, topical and/or gastrointestinal concentrations that have been found to be effective in animals. One skilled in the art can determine the effective amount for subject use, especially in light of the animal model experimental data described herein. Based on animal data, and other types of similar data (such as in vitro data), those skilled in the art can determine the effective amounts of compositions of the present disclosure.

The effective amount when referring to an agent (e.g., nucleic acid sequence or polypeptide) or combination of agents can generally mean the dose ranges, modes of administration, formulations, etc., that have been recommended or approved by any of the various regulatory or advisory organizations in the medical or pharmaceutical arts (e.g., FDA, AMA) or by the manufacturer or supplier.

The Examples below describe the optimization of the vaccines and methods disclosed herein. The Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

Without being bound by a specific mechanism, the disclosure teaches that iDC can manifest enhanced interaction with antigen, when iDC are exposed to an increased level of an immune-cell targeting molecule, such as the chemokine MIP-3α. MIP-3α binds to chemokine receptor CCR6, and the underlying concept of CCR6-mediated reaction to antigen by the DC means that other CCR6 ligands, and ligands to other chemokine receptors on DCs as described herein, are suitable for achieving the purposes described herein.

The data discussed in the Examples provide compelling evidence of an unexpected synergistic, or multiplying, effect achieved by administering the vaccine with adjuvant as described. The development of a successful antibody neutralization response in mammals, and a correlating reduction in liver-stage infection by challenge sporozoites, advances the field of malaria vaccines and hastens the eventual protection of subjects, including humans, from infection. These results will find application for antigens of other infectious agents.

### Examples

In the examples, differences among groups were analyzed by an ANOVA test (Stata Corp). Value of p<0.05 was considered to be significant. Where indicated, P. yoelii parasites were used for challenge. Sporozoites were obtained by hand dissection of infected mosquito salivary glands. The isolated sporozoites were suspended in HBSS medium containing 1% normal mouse serum. All challenges were accomplished by injecting 5×10³ sporozoites in the tail vein. Humoral immune responses to the immunodominant B cell epitope was measured using variants of CSP-specific ELISA assays (Del Giudice et al., Bull. World. Health Org. 67:515-523, 1989 which is incorporated by reference herein for its teachings regarding the same).

### Example 1

### Expression of Construct of Malaria DNA Vaccine Candidate and Controls

In this and following examples, abbreviations are used as follows: M refers to MIP-3α; CSP refers to a segment of the P. CSP (from P. yoelli or P. falciparum, as indicated in the specific methods); and MCSP refers to a fusion protein of MIP-3α and CSP.

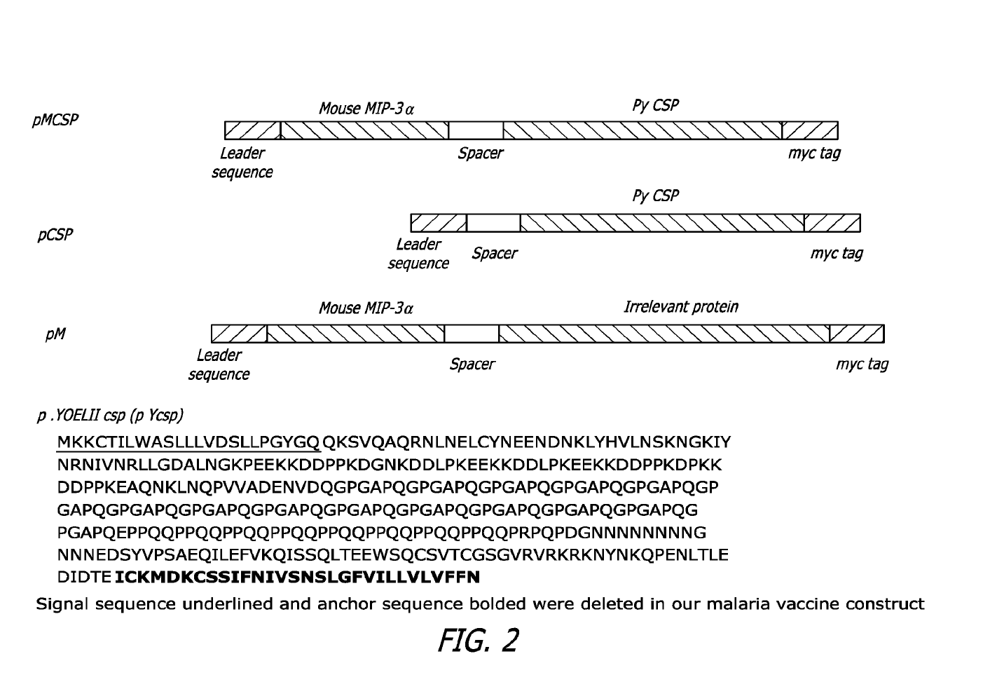

A DNA construct (pMCSP) encoding mouse MIP-3α fused with the fragment of CSP with deletion of N-terminal signaling sequence (21aa) and C-terminal anchor region (73aa) is described in FIG. 2. Control plasmids were similarly constructed: DNA constructs identical to the experimental constructs, except lacking the MIP-3α fusion component (pCSP) and a mouse MIP-3αcontaining plasmid (pM) in which MIP-3α is fused to DNA encoding an irrelevant epitope. Plasmids were purified using Endofree purification columns (Qiagen, Hilden, Germany) and stored at −20° C. in PBS.

Expression of MIP-3α, CSP and MCSP fusion protein following in vitro transfection was detected with anti-myc tag antibody. The constructs expressed MCSP, CSP, and MIP-3α, in either cells or supernatant fraction, respectively (FIG. 3). MCSP and CSP were also recognized by antibody against major immunodominant repeats of CSP. Expression of MIP-3α, did not result in changed expression for CSP.

This example establishes that DNA encoding a chemokine and DNA encoding a malaria antigen can be expressed together to yield a fusion protein, without diminishing the expression of the antigen. This is important in order to ensure adequate antigen availability for uptake by the DCs upon binding the ligand or chemokine (here, MIP-3α) to a receptor on the DCs. These results also indicate that the enhanced response to the chemokine construct, with or without adjuvant, was not simply attributable to enhanced expression of the chemokine construct compared to the CSP construct without the chemokine.

### Example 2

### ELISA for Antibody Response to CSP DNA Vaccine Candidates in C57BL/6 Mice

Six- to eight-week-old female C57BL/6 (H-2b) mice were obtained from The Jackson Laboratory (Bar Harbor, Me.) and maintained in a pathogen-free micro-isolation facility in accordance with the National Institutes of Health guidelines for the humane use of laboratory animals. Vaxfectin® formulations were prepared by adding 2 ml of 0.9% NaCl solution to 2.18 mg of Vaxfectin®, then mixing the same volumes of 1 mg/ml DNA and Vaxfectin®, and diluting the mixture to the desired concentration with PBS.

The antibody response to DNA vaccine candidates was first evaluated in C57BL/6 mice. C57BL/6 mice were immunized with 2 μg of the constructs, which were delivered as a single injection in 100 μl of PBS or PBS formulated with Vaxfectin®. Mice received three immunizations at bi-weekly intervals. For the positive control group, 10⁵ (initial immunization) and 5×10⁴ (booster immunizations) irradiated P. yoelii sporozoites (17×N) obtained from Anopheles stephensi were inoculated by tail-vein injection at the same time-points. Mice were bled two weeks after final immunization to determine specific antibody concentrations (FIG. 4).

Significantly higher antibody titers were induced in the mice immunized with Vaxfectin®-formulated pMCSP as compared to the mice immunized with Vaxfectin®-formulated pCSP, unformulated pMCSP, and pCSP. The antibody response to Vaxfectin®-formulated pMCSP was similar to the antibody response induced by irradiated P. yoelii sporozoites. No antibody response was detected to MIP-3α, which was used as negative control. This result shows that fusion with MIP-3αplus administration with the adjuvant Vaxfectin® enhanced the humoral response to malaria antigen CSP in a synergistic manner, compared with a simple additive effect that might have been expected based on the activity of each component alone.

In FIG. 4, histograms represent means±the standard error of the mean (n=5) of antibody concentrations. The antibody response to MIP-3α-Vaxfectin®, CSP, and MCSP were all significantly lower than the antibody response to the fusion construct vaccine (MCSP/Vaxfectin®) or to IrSpz (FIG. 4).

In summary, the data in Example 2 demonstrate that the DNA vaccine construct of MIP-3α and antigen CSP was not only capable of raising an antibody response in mice, the antibody response to the fusion construct vaccine in combination with the adjuvant was comparable to the “gold standard” for malaria vaccine, IrSpz.

### Example 3

### Enhancement of Protection by Formulation of pMCSP in Vaxfectin®

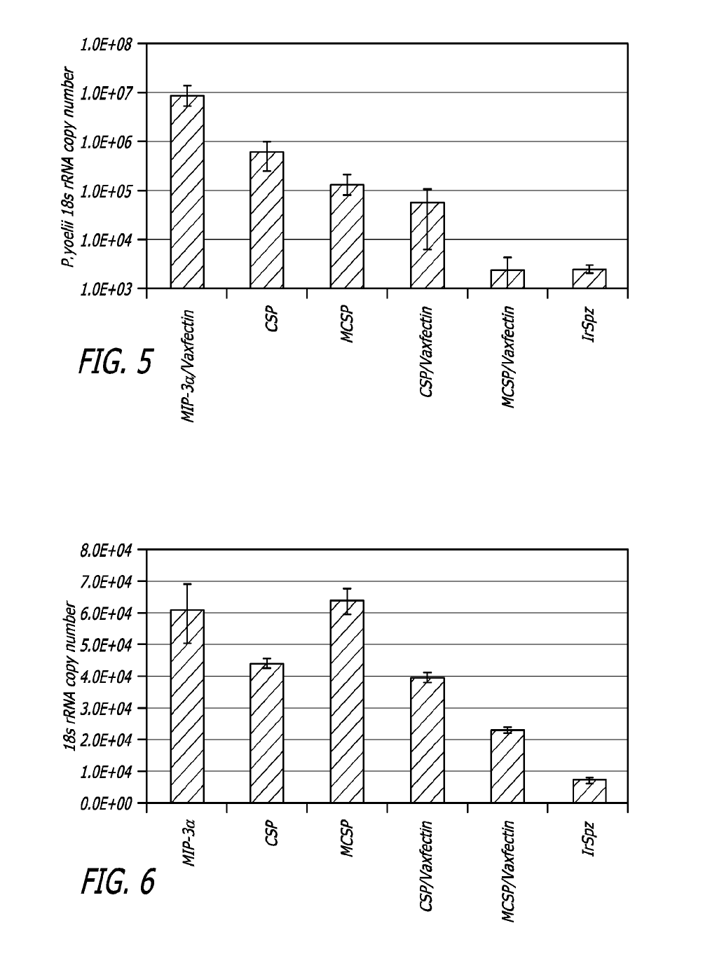

In order to investigate the protection by pMCSP immunization in vivo, a challenge experiment was performed by inoculating 5×10³ P. yoelii sporozoites into the mice that had been immunized with various DNA constructs and IrSpz (as above) two weeks after the last immunization. 48 h after infection, parasite-specific 18S rRNA levels in the liver were determined by qRT-PCR, as shown in FIG. 5. The lowest infection was observed in the mice immunized with IrSpz. The mice immunized with negative control pM acquired the highest infection. The MCSP plus Vaxfectin® group was essentially equivalent to the irradiated sporozoite group and differed from the other groups receiving CSP (with either Vaxfectin® alone or MIP-3αalone) by between approximately 1.5 to 2 orders of magnitude.

These results support the conclusion that MIP-3α fusion combined with Vaxfectin® effected an enhancement of the DNA vaccine's protection in a synergistic manner, greater than a simple additive result. The data in FIG. 5 are expressed as mean±s.d. of copy numbers from 5 mice per group.

### Example 4

### In Vitro Neutralization Assay

A goal of this disclosure is to provide antibody specific for CSP that can protect humans against malaria infection. To assess biological activity of specific antibodies induced by DNA vaccine, this example was performed to investigate the inhibitory capacity of anti-CSP sera in an in vitro neutralization assay.

Sporozoite neutralization assay method: The assay was performed in a total volume of 100 μl that contained 1×10⁵ sporozoites in dissection medium, and 10 μl of each immune serum from immunized mice. The sporozoite mixtures were incubated for 40 min on ice. The sporozoites were then added to HepG1.6 cell cultures that were maintained at 37° C. in 5% CO₂. The incubation was carried out for 48 h with changes of culture media every 24 h. All neutralization assays were performed in duplicates. At the end of 48 hours, the cells were harvested. Total RNA was isolated and reverse transcription was performed. 18S rRNA were detected and quantified by real-time PCR. To perform the experiments of this Example, sera from immunized mice as in Example 2 were incubated with live P. yeolii sporozoites for 40 min on ice and then added to HepG1.6 cell cultures that were maintained at 37° C. in 5% CO₂. 48 h after infection, parasite replication was determined by quantitative RT-PCR (qRT-PCR) measurement of P. 18S ribosomal RNA in total RNA extracted from infected cells. Results were normalized for RNA recovery by qRT-PCR measurement of cellular actin mRNA in the same samples.

In this experiment, sera from sporozoite-immunized mice reduced sporozoite infectivity 3-10 fold compared to sera from DNA plasmid-immunized mice. However, sera from the mice that were immunized with Vaxfectin®-formulated pMCSP reduced sporozoites infectivity 1.7-5 fold compared to sera from the mice that were immunized with Vaxfectin®-formulated pCSP, unformulated pMCSP and pCSP. A significant increase of protection was observed between sera from mice immunized with pMCSP and pCSP that were all formulated with Vaxfectin® (FIG. 6).

This example shows that the protective ability of specific antibody against CSP can be increased by fusing MIP-3α with CSP, formulated with the adjuvant Vaxfectin®.

### Example 5

### T Cell Depletion Did not Change the Protection of Vaxfectin®-Formulated-pMCSP

This Example was performed in order to determine whether T cells confer protection on immunization with Vaxfectin®-formulated pMCSP.

T-cell depletion was performed as follows: To deplete the CD4+, CD8+, or both T cell subsets, immunized mice were injected intraperitoneally (i.p.) with anti-CD4, anti-CD8 or both mAbs. Each mouse received daily doses of 200 μg of anti-CD4 or anti-CD8 or both antibodies for two days. 24 h after the last immunization, the efficacy of the depletion was estimated by two-color flow cytometry analysis of peripheral blood lymphocytes, using FITC-conjugated anti-CD4 or APC-conjugated anti-CD8 mAbs.

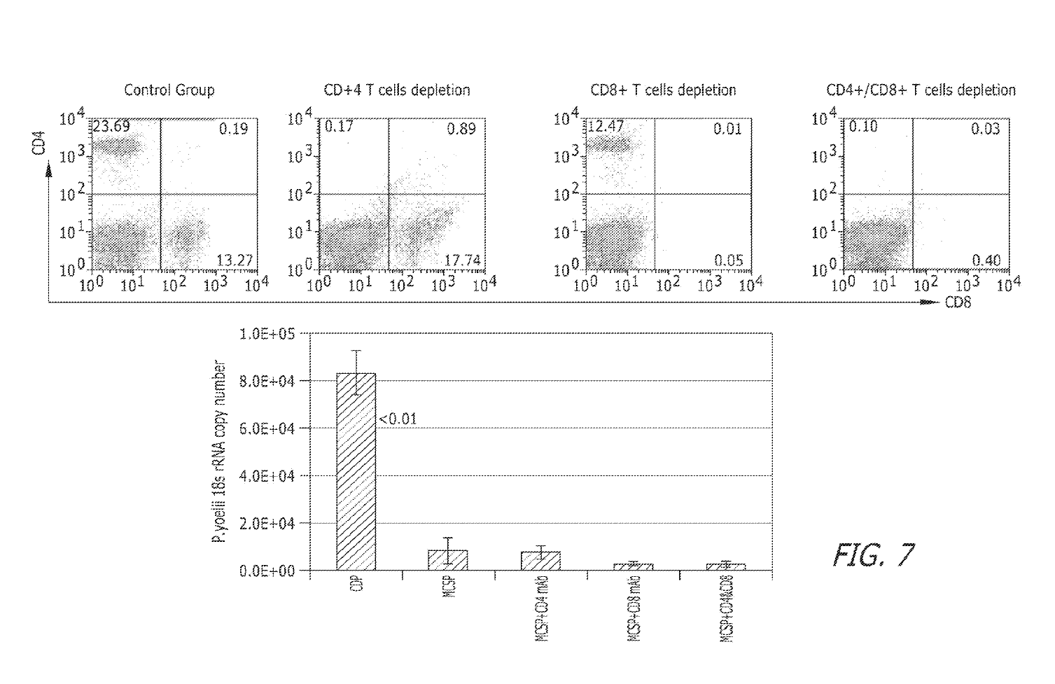

C57BL/6 mice were immunized with 2 μg of pMCSP construct, and were delivered as single injection in 100 μl of PBS formulated with Vaxfectin®. Mice received three immunizations at bi-weekly intervals. To deplete the CD4+, CD8+, or both T cell subsets, immunized mice were injected i.p with anti-CD4, anti-CD8 or both mAbs 3 days before challenge. Mice receiving rat IgG antibody were used as negative control. 24 hours later, the efficacy of the depletion was estimated by two-color flow cytometry analysis of peripheral blood lymphocytes using FITC-conjugated anti-CD4 or APC-conjugated anti-CD8 mAbs (FIG. 7 top panel). Data show the CD4 and CD8 expression on combined peripheral lymphocytes of three mice in each group.

Sporozoite challenge was performed by injecting 2500 sporozoites in mouse tail veins 14 days after the final booster immunization. As a control for the anti-CD4 and anti-CD8 antibody treatments, mice not receiving those antibodies were administered a control IgG serum. Not noted on the FIG. is that all immunization groups in this experiment received Vaxfectin in addition to the indicated plasmids.

After sporozoite challenge, parasite replication was determined by qRT-PCR measurement of P. 18S ribosomal RNA in total RNA extracted from liver cells (FIG. 6 bottom panel). Parasite replication in the liver of all animals treated with rat IgG were similar with that of all mice depleted of CD4, CD8, and both CD4 and CD8 T cells (p>0.05). This observation indicates that enhanced protection of Vaxfectin®-formulated pMCSP is not primarily mediated by T-cell-effector mechanisms, which has important implications for the vaccine's mechanism of action.

### Example 6

### Real-Time PCR Evaluation of Expression Levels of Cytokines

Vaxfectin® can upregulate some cytokines, chemokines and Toll-like receptor pathway transcripts at the site of administration. This Example was performed to determine whether or not MIP-3α can modulate this upregulation by Vaxfectin® and contribute to the enhancement of protection of Vaxfectin®-formulated pCSP. Methods used in this example: Total RNA was isolated from tissue samples (n=3 per group). Reverse transcription was performed using total RNA (5 μg) as template and High-Capacity cDNA reverse transcription kit (Applied Biosystems, Foster City, Calif.) reagents. Relative transcript levels for the proteins as listed below were determined in tissue samples collected 24 h and 48 h after vaccination. For this purpose commercially available TaqMan® Gene Expression Assays (Applied Biosystems) were used. These markers were selected based on preliminary exploratory work on biomarkers associated with Vaxfectin® formulations. Analysis was performed using the ABI Prism® 7900HT Sequence Detection System (Applied Biosystems) and cycling conditions suggested by the manufacturer. A comparative method was used to determine relative quantitation. Ct values for target gene amplification were normalized by subtracting the Ct values for the housekeeping gene GAPDH.

To address this question of cytokine expression, 2 μg of Vaxfectin®-formulated pMCSP was inoculated into C57BL/6 mice tibialis anterior muscles, and then cytokine levels of muscle samples were analyzed by Real-time PCR. Levels of IL-2, IL-6, IL-10, IL-12β, IFN-γ, TNF-α, CCL2, CXCL2, CXCL9, TGF-β and MIP-3α were evaluated 24 h and 48 h after injection, and the results are shown in FIGS. 8A and 8B. There was no significant difference between pMCSP immunized mice and pCSP immunized mice at indicated time points, in terms of the pattern of cytokines at the site of inoculation. The results imply that the combined effects of MIP-3α and Vaxfectin® might not be due to cytokine and chemokine production.

### Example 7

### Ability of Vaxfectin®-Formulated Plasmid to Attract Infiltrates Including DCs into Injected Muscles

To determine whether iDC-specific chemotactic factor fused DNA vaccine with adjuvant Vaxfectin® would lead to increased recruitment of DCs at the site of inoculation, pMCSP and pCSP were injected in C57BL/6 mice tibialis anterior muscles with or without Vaxfectin® formulation. Methods used in this Example: Immunized mice muscles were harvested, minced, and incubated with digestion buffer (Hanks' Balanced Salt Solution [Gibco], supplemented with 0.5% collagenase, 0.2% BSA, and 0.025% trypsin) for 30 minutes at 37° C. with vortexing every 5 minutes to facilitate tissue dissociation. Cell suspensions were filtered, then washed twice with PBS containing 2% FBS. The cells numbers were counted and 1×10⁶ cells were incubated with MIP-3α fusion protein and the protein without MIP-3α for 30 min on ice, and stained with PerCP-cy5.5-labeled CCR6 and APC-labeled CD11c antibodies.

After 24 h, injected muscles were harvested, minced and digested as described above. Extracted cells were counted and then were used for flow cytometry to detect CD11c+ cells. The results are shown in Table 4. The use of Vaxfectin® plus either CSP or MCSP resulted in increased cell numbers. Adding MIP-3αdid not result in an increase compared to Vaxfectin® plus CSP, but both were higher than the other controls. The data suggest that Vaxfectin® enhances the recruitment of cells, including DC's, in the presence of antigen. The data also suggest that the addition of MIP-3α to the vaccine construct did not enhance the recruitment of dendritic cells compared to the CSP/Vaxfectin combination. The data are consistent with MIP-3αacting primarily by increasing the efficiency of antigen binding to the DCs.

TABLE 4

| | Total cell | | CD11c+ cell numbers |
|---|---|---|---|
| Candidate vaccine | numbers | CD11c+ | in total cells |
| or control | (10⁴) | Cells | (10⁴) |
| Saline | 34 | 6.82% | 0.95 |
| Vaxfectin ® (2 μg) | 34.5 | 6.83% | 1.12 |
| MIP3α/Vaxfectin ® | 32 | 6.60% | 1.16 |
| (2 μg/2 μg) | | | |
| CSP (2 μg) | 33 | 7.08% | 1.00 |
| CSP/Vaxfectin ® | 89 | 7.74% | 3.69 |
| (2 μg/2 μg) | | | |
| MCSP (2 μg) | 29 | 9.09% | 1.09 |
| MCSP/Vaxfectin ® | 89.5 | 8.32% | 3.18 |
| (2 μg/2 μg) | | | |

### Example 8

### Antibody Response to P. falciparum CSP in C57BL/6 Mice

C57BL/6 mice were immunized with P. falciparum CSP. As shown in FIG. 8, antibody response at 4 and 6 weeks was increasingly greater for the hMfCSP/Vaxfectin® composition than for the hMIP-3α, Vaxfectin®, fCSP, fCSP/Vaxfectin®, or hMfCSP. For the 6-week data, P <0.01 compared to the other groups, indicating that this increase was statistically significant.

### Example 9

### Transgenic P Berghei Replication in C57BL/6 Mice: Liver

Challenge with 5000 sporozoites. C57BL/6 mice were vaccinated with controls and the test vaccine hMfCSP/Vaxfectin®. The mice were then challenged with 5000 sporozoites from transgenic P. berghei, a mouse P. parasite engineered to express P. falciparum CSP. P. falciparum is the form of malaria that is most lethal for humans. Liver infection was measured by assaying 18s rRNA copy numbers. The lower the copy number, the more effective the vaccine.

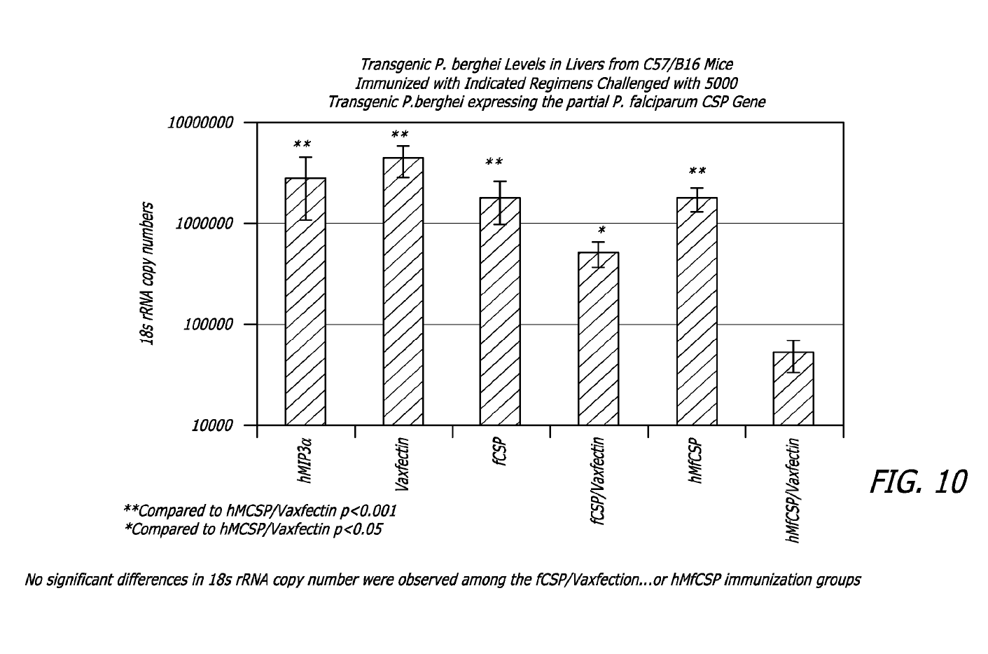

As shown in FIG. 10, there was a statistically significant reduction in liver infection for the hMfCSP/Vaxfectin® vaccine compared to all the other groups: hMIP-3α; Vaxfectin®; fCSP; fCSP/Vaxfectin®; and hMfCSP. These results confirm that administration of a vaccine consisting of DNA encoding human P. falciparum circumsporozoite antigen linked to DNA encoding the chemokine MIP-3α, administered with the adjuvant Vaxfectin®, raised an immune response in mice to subsequent challenge with sporozoites carrying the human CSP antigen. Importantly, the immune response successfully reduced liver stage infection.

### Example 10

### Neutralizing Antibody Response of Aotus Monkeys to hMfCSP/Vaxfectin® Vaccine

As discussed in the Detailed Description, Aotus monkeys are a suitable animal model for human malaria infection and for testing vaccines. Five monkeys received four injections of the adjuvant Vaxfectin® and 500 μg of DNA at a molar ratio of 1:4 (DNA:Vaxfectin®) at two week intervals. Immunization with experimental or control preparations were delivered intradermally (up to 100 μl/site). The two experimental groups were (1) MIP-3α fused PfCSP (P. falciparum circumsporozoite-encoding-DNA plus Vaxfectin®); and (2) PfCSP-DNA plus Vaxfectin®.

Prior to each immunization, 3 ml of venous blood was obtained from each monkey and another blood sample was obtained two weeks following the final immunization. These blood samples were used to measure specific antibody responses, and serum was used for in vitro challenge assays against a transgenic mouse malaria strain expressing the P. falciparum CSP.

As described above, monkeys were vaccinated with controls and hMfCSP/Vaxfectin® vaccine construct, in four injections given at 2-week intervals. Monkeys injected with hMfCSP/Vaxfectin® demonstrated increasing antibody titer over the course of injection, compared to CSP without MIP-3α. The sera of two of these monkeys, designated 6160 and 6166, were chosen for further study.

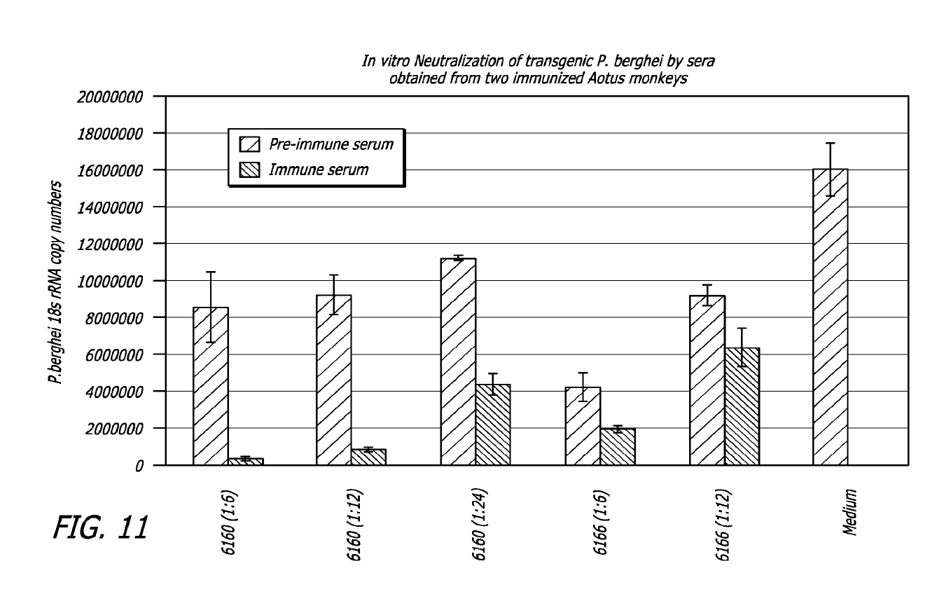

To test the ability of the antibodies to specifically neutralize malaria at the early stage of infection (sporozoites expressing CSP), sera from monkeys 6160 and 6166 were used in a neutralization assay in HEP62 liver cells. Compared to pre-immunization serum, the sera from monkeys 6160 and 6166 significantly neutralized transgenic P. berghei replication in vitro, as measured by P. berghei 18s rRNA copy number in the HEP62 cells. (FIG. 11.)

### Example 11

### Electroporation for Vaccine Delivery

For DNA immunization an in vivo electroporation system, termed Easy Vax, developed by Cyto Pulse, Baltimore, Md., was used. (Harvard Apparatus, a division of Harvard Bioscience, Inc., Holliston, Mass.) This method and apparatus are not intended to be limiting, and any electroporation system suitable for clinical use can be substituted. C57BL/6 mice were immunized with 2 μg of pDNA, with or without adjuvant (Vaxfectin®) or electroporation. Mice received one of the following vaccines or controls: MIP3α/Vaxfectin®; CSP/electroporation; MCSP/electroporation; CSP; MCSP; CSP/Vaxfectin®; or MCSP/Vaxfectin®. IrSpz also served as a control.

The results are shown in FIG. 12. Of the vaccine groups using the fusion vaccine MCSP, electroporation was comparable (in antibody response to CSP) to Vaxfectin® and to IrSpz. The data supports use of electroporation to successfully deliver DNA encoding the fusion protein.

Following immunization, the mice immunized with these groups of vaccines and controls were challenged with 5000 P. yoelli sporozoites. As shown in FIG. 12, the P. yoelli 18s rRNA copy numbers in liver were lower for the MCSP/electroporation group than for the CSP/electroporation group.

### Example 12

### Bloodstream Malaria Levels Following Vaccination

First, IgG levels following immunization with constructs were compared along with a negative control. The negative control was PBS. One vaccine is represented by the fCSP-Vaxfectin® group. Another vaccine is represented by the hMfCSP group. The approach of the disclosed methods is represented by the hMfCSP/Vaxfectin® group, which yielded a significantly higher level of antibody response. The numerical results are as follows in Table 5.

TABLE 5

| Group | IgG Concentration (μg/mL) |
|---|---|
| PBS (negative control) | 0 |
| fCSP/Vaxfectin ® | 11.1 |
| hMfCSP | 105.3 |
| hMfCSP/Vaxfectin ® | 1638.9 |

Protection against bloodstream malaria, the symptomatic and potentially lethal form of the disease, was evaluated. Transgenic P. berghei sporozoites carrying P. falciparum CSP were used for challenge. Sporozoites were obtained by hand dissection of salivary glands of Anopheles stephensi mosquitoes. The isolated sporozoites were suspended in HBSS medium containing 1% normal mouse serum. Challenges to evaluate protection from blood stage malaria were accomplished by injecting 1×10³ sporozoites in the tail vein.

Differences among groups were analyzed by a Fisher exact. A value of p <0.05 was considered to be significant.

The results in Table 6 show that the fCSP/Vaxfectin® approach did not protect any mice from bloodstream malaria, performing no better than the negative control (PBS). The hMfCSP approach protected 20% of the mice from bloodstream malaria. Showing greater protection than these, the vaccine of the disclosed methods protected 80% of mice from bloodstream malaria.

TABLE 6

| | No. of infected mice/ | |
|---|---|---|
| Immunization | No. of challenged mice | % Protection |
| PBS (negative control) | 4/4 | 0% |
| fCSP/Vaxfectin ® | 5/5 | 0% |
| hMfCSP | 4/5 | 20% |
| hMfCSP/Vaxfectin ® | 1/5 | 80% |

Differences in protection against bloodstream malaria were assessed in a separate experiment. In this experiment, PBS and hMIP3α/Vaxfectin® (no antigen) represent negative controls while fCSP/Vaxfectin®, the DNA construct without the hMIP3αrepresents another approach and hMfCSP/Vaxfectin® represents the approach of the disclosed methods. The difference between 4/5 infected/challenged mice and 0/5 infected/challenged mice was statistically significant at p<0.05. The results summarized in Table 7 show that the hMIP3α/Vaxfectin® approach protected only 40% of challenged mice from bloodstream malaria. In contrast, the approach of the disclosed methods protected 100% of challenged mice from bloodstream malaria.

TABLE 7

| | No. of infected mice/ | |
|---|---|---|
| Immunization | No. of challenged mice | % Protection |
| PBS (negative control) | 4/5 | 20% |
| hMIP3α/Vaxfectin ® | 4/5 | 20% |
| (negative control) | | |
| fCSP/Vaxfectin ® | 2/5 | 40% |
| hMfCSP/Vaxfectin ® | 0/5 | 100% |

Taken together, these Examples demonstrate that the DNA vaccine hMfCSP/Vaxfectin® was successful in raising malaria sporozoite-specific antibodies in mice and non-human primates, and that the antibodies significantly reduced challenge malaria parasite replication in liver cells in vivo. Without being bound by a specific mechanism, the data show that enhanced efficacy was uniquely attributable to the combined use of the adjuvant and the chemokine fusion construct. Thus, the vaccines disclosed herein provide an advance over existing vaccines currently in use and are suitable for further testing in humans.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

1. A vaccine comprising a DNA plasmid comprising a polynucleotide encoding (i) an antigenic polypeptide; and (ii) at least one ligand for an immature dendritic cell receptor, wherein said DNA encoding the antigenic polypeptide and the DNA encoding the ligand are linked by a polynucleotide linker, and wherein said vaccine comprises at least one adjuvant.

2. The nucleic acid vaccine of claim 1 wherein said antigen polypeptide is a Plasmodium (P.) polypeptide.

3. The vaccine of claim 2 wherein said P. protein is a protein expressed by a P. species that infects humans.

4. The vaccine of claim 3 wherein said P. species is P. falciparum, P. vivax, P. ovale or P. malariae.

5. The vaccine of claim 1 wherein said antigen polypeptide is P. circumsporozoite protein (CSP) or an antigenic fragment or mimic thereof.

6. The vaccine of claim 1 wherein said ligand binds to the CCR6 receptor expressed on the immature dendritic cell.

7. The vaccine of claim 6 wherein said ligand is a chemokine.

8. The vaccine of claim 7 wherein said chemokine is MIP-3α.

9. The vaccine of claim 6 wherein said ligand is a human β-defensin or a viral β-defensin.

10. The vaccine of claim 1 wherein said adjuvant comprises a cationic lipid and a neutral phospholipid in an aqueous vehicle.

11. The vaccine of claim 1 wherein the plasmid DNA is in a circular plasmid form, wherein the plasmid additionally comprises an origin of replication, a promoter, and a transcription termination sequence.

12. A method of enhancing the efficacy of a plasmid DNA vaccine comprising DNA encoding (i) an antigenic polypeptide; and (ii) at least one ligand for an immature dendritic cell receptor, said method comprising adding to the plasmid DNA vaccine an adjuvant comprising a cationic lipid and a neutral phospholipid in an aqueous vehicle.

13. A method of preventing liver-stage malaria infection in a subject at risk of malaria infection, said method comprising administering to said subject a DNA vaccine of claim 1.

14. The method of claim 13 wherein said vaccine is administered to the subject by injection, for example intradermal or intramuscular

15. A method of preventing liver-stage malaria infection, said method comprising administering to a subject at risk of malaria infection a DNA vaccine comprising a plasmid DNA containing and expressing in vivo a polynucleotide encoding (i) an antigenic polypeptide; and (ii) at least one ligand for a dendritic cell receptor, wherein said DNA encoding the antigenic polypeptide and the DNA encoding the ligand are linked by a polynucleotide linker, wherein said vaccine is administered to the skin by electroporation.

16. The method of claim 15 wherein said antigen polypeptide is a P. polypeptide.

17. The method of claim 16 wherein said P. polypeptide is a polypeptide expressed by a P. species that infects humans.

18. The method of claim 17 wherein said P. species is P. falciparum, P. vivax, P. ovale, or P. malariae.

19. The method of claim 16 wherein said antigen polypeptide is P. CSP or an antigenic fragment or mimic thereof.

20. The method of claim 15 wherein said ligand binds to the CCR6 receptor expressed on an immature dendritic cell.

21. The method of claim 15 wherein said ligand is a chemokine.

22. The method of claim 21 wherein said chemokine is MIP-3α.

23. The method of claim 15 wherein said ligand is a human β-defensin or a viral β-defensin.

24. The method of claim 15 wherein said vaccine is administered more than once.

25. The method of claim 15 wherein the antibody titer to the antigen in the blood is measured after vaccination to determine the need for additional vaccine administration.

26. A method of reducing the bloodstream malaria levels in a subject at risk of malaria infection, said method comprising administering to said subject a DNA vaccine of claim 1.

27. The method of claim 26 wherein said vaccine is administered to the subject by injection.

28. The method of claim 26 wherein said vaccine is administered to the subject by electroporation and in combination with an adjuvant.


SEQUENCE LISTING (32 sequences)

SEQ ID NO: 1 - protein, 369 aa, Plasmodium yoelii
MKKCTILWASLLLVDSLLPGYGQQKSVQAQRNLNELCYNEENDNKLYHVLNSKNGKIYNRNIVNRLLGDALNGKPEEKKDDPPKDGNKDDLPKEEKKDDLPKEEKKDDPPKDPKKDDPPKEAQNKLNQPWADENVDQGPGAPQGPGAPQGPGAPQGPGAPQGPGAPQGPGAPQGPGAPQGPGAPQGPGAPQGPGAPQGPGAPQGPGAPQGPGAPQGPGAPQGPGAPQEPPQQPPQQPPQQPPQQPPQQPPQQPPQQPPQQPRPQPDGNNNNNNNNGNNNEDSYVPSAEQILEFVKQISSQLTEEWSQCSVTCGSGVRVRKRKNYNKQPENLTLEDIDTEICKMDKCSSIFNIVSNSLGFVILLVLVFFN

SEQ ID NO: 2 - DNA, 1381 bp, Artificial Sequence
  Description of Artificial Sequence hMfCSP fusion protein
ctgcagtcaccgtcgtcgacagagctgagatcctacaggagtccagggctggagagaaaacctctgcgaggaaaaggaaggagcaagccgtgaatttaagggacgctgtgaagcaatcatggatgcaatgaagagagggctctgctgtgtgctgctgctgtgtggagcagtcttcgtttcgcccagcggtaccggatccgcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatggaattcaacgacgctcaggcgccgaagagtggatcctctagaatggagtaccagtgctacggctcctcctccaacacccgcgtgctgaacgagctgaactacgacaacgccggcaccaacctgtacaacgagctggagatgaactactacggcaagcaggagaactggtactccctgaagaagaactcccgctccctgggcgagaacgacgacggcaacaacgaggacaacgagaagctgcgcaagcccaagcacaagaagctgaagcagcccgccgacggcaaccccgaccccaacgccaaccccaacgtggaccccaacgccaaccccaacgtggaccccaacgccaaccccaacgtggaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgtggaccccaacgccaaccccaacgccaaccccaacaagaacaaccagggcaacggccagggccacaacatgcccaacgaccccaaccgcaacgtggacgagaacgccaacgccaactccgccgtgaagaacaacaacaacgaggagccctccgacaagcacatcaaggagtacctgaacaagatccagaactccctgtccaccgagtggtccccctgctccgtgacctgcggcaacggcatccaggtgcgcatcaagcccggctccgccaacaagcccaaggacgagctggactacgccaacgacatcgagaagaaaatctgcaagatggagaagtgcagatccgcagaagaacagaaactgatctcagaagaggatctgtgatctagaagatct

SEQ ID NO: 3 - DNA, 199 bp, Homo sapiens
ctgcagtcaccgtcgtcgacagagctgagatcctacaggagtccagggctggagagaaaacctctgcgaggaaaaggaaggagcaagccgtgaatttaagggacgctgtgaagcaatcatggatgcaatgaagagagggctctgctgtgtgctgctgctgtgtggagcagtcttcgtttcgcccagcggtaccggatcc

SEQ ID NO: 4 - DNA, 213 bp, Homo sapiens
gcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatg

SEQ ID NO: 5 - DNA, 42 bp, Artificial Sequence
  Description of Artificial Sequence Spacer sequence
gaattcaacgacgctcaggcgccgaagagtggatcctctaga

SEQ ID NO: 6 - DNA, 927 bp, Plasmodium falciparum
atggagtaccagtgctacggctcctcctccaacacccgcgtgctgaacgagctgaactacgacaacgccggcaccaacctgtacaacgagctggagatgaactactacggcaagcaggagaactggtactccctgaagaagaactcccgctccctgggcgagaacgacgacggcaacaacgaggacaacgagaagctgcgcaagcccaagcacaagaagctgaagcagcccgccgacggcaaccccgaccccaacgccaaccccaacgtggaccccaacgccaaccccaacgtggaccccaacgccaaccccaacgtggaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgtggaccccaacgccaaccccaacgccaaccccaacaagaacaaccagggcaacggccagggccacaacatgcccaacgaccccaaccgcaacgtggacgagaacgccaacgccaactccgccgtgaagaacaacaacaacgaggagccctccgacaagcacatcaaggagtacctgaacaagatccagaactccctgtccaccgagtggtccccctgctccgtgacctgcggcaacggcatccaggtgcgcatcaagcccggctccgccaacaagcccaaggacgagctggactacgccaacgacatcgagaagaaaatctgcaagatggagaagtgcagatccgcagaagaacagaaactgatctcagaagaggatctgtgatctagaagatct

SEQ ID NO: 7 - protein, 416 aa, Artificial Sequence
  Description of Artificial Sequence hMfCSP fusion protein
MDAMKRGLCCVLLLCGAVFVSPSGTGSAASNFDCCLGYTDRILHPKFIVGFTRQLANEGCDINAIIFHTKKKLSVCANPKQTWVKYIVRLLSKKVKNMEFNDAQAPKSGSSRMEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCRSAEEQKLISEEDL

SEQ ID NO: 8 - protein, 10 aa, Artificial Sequence
  Description of Artificial Sequence Linker sequence
EFNDAQAPKS

SEQ ID NO: 9 - DNA, 1381 bp, Artificial Sequence; identical to SEQ ID NO: 2
  Description of Artificial Sequence hMfCSP Sequence

SEQ ID NO: 10 - protein, 4 aa, Artificial Sequence
  Description of Artificial Sequence Repeat
NANP

SEQ ID NO: 11 - protein, 4 aa, Artificial Sequence
  Description of Artificial Sequence Repeat
NVDP

SEQ ID NO: 12 - protein, 4 aa, Artificial Sequence
  Description of Artificial Sequence Repeat
NPDP

SEQ ID NO: 13 - DNA, 336 bp, Homo sapiens
agactcagctcctggtgaagctcccagccatcagccatgagggtcttgtatctcctcttctcgttcctcttcatattcctgatgcctcttccaggtgtttttggtggtataggcgatcctgttacctgccttaagagtggagccatatgtcatccagtcttttgccctagaaggtataaacaaattggcacctgtggtctccctggaacaaaatgctgcaaaaagccatgaggaggccaagaagctgctgtggctgatgcggattcagaaagggctccctcatcagagacgtgcgacatgtaaaccaaattaaactatggtgtccaaagatacgca

SEQ ID NO: 14 - protein, 64 aa, Homo sapiens
MRVLYLLFSFLFIFLMPLPGVFGGIGDPVTCLKSGAICHPVFCPRRYKQIGTCGLPGTKCCKKP

SEQ ID NO: 15 - DNA, 437 bp, Homo sapiens
caaatccatagggagctctgccttaccattgggttcctaattaactgagtgagtgggtgtgttctgcatggtgagaggcattggaatgatgcatcagaaaacatgtcataatgtcatcactgtaatatgacaagaattgcagctgtggctggaacctttataaagtgaccaagcacaccttttcatccagtctcagcgtggggtgaagcctagcagctatgaggatccattatcttctgtttgctttgctcttcctgtttttggtgcctgttccaggtcatggaggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataaaaaccctgaaacatg

SEQ ID NO: 16 - protein, 67 aa, Homo sapiens
MRIHYLLFALLFLFLVPVPGHGGIINTLQKYYCRVRGGRCAVLSCLPKEEQIGKCSTRGRKCCRRKK

SEQ ID NO: 17 - DNA, 851 bp, Homo sapiens
agaatataacagcactcccaaagaactgggtactcaacactgagcagatctgttctttgagctaaaaaccatgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaagctattttttaaaaaaaactatttaacattcttttgtttatattgttttgtctcctaaattgttgtaattgcattataaaataagaaaaatattaataagacaaatattgaaaataaagaaacaaaaagttcttctgttaaaaaaaaa

SEQ ID NO: 18 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 19 - DNA, 848 bp, Homo sapiens
agaatataacagcactcccaaagaactgggtactcaacactgagcagatctgttctttgagctaaaaaccatgtgctgtaccaagagtttgctcctggctgctttgatgtcagtgctgctactccacctctgcggcgaatcagaagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaagctattttttaaaaaaaactatttaacattcttttgtttatattgttttgtctcctaaattgttgtaattgcattataaaataagaaaaatattaataagacaaatattgaaaataaagaaacaaaaagttcttctgttaaaaaaaaa

SEQ ID NO: 20 - DNA, 852 bp, Mus musculus
gagcactcgcagggcactgggtacccagcactgagtacatcaactcctggagctgagaatggcctgcggtggcaagcgtctgctcttccttgctttggcatgggtactgctggctcacctctgcagccaggcagaagcagcaagcaactacgactgttgcctctcgtacatacagacgcctcttccttccagagctattgtgggtttcacaagacagatggccgatgaagcttgtgacattaatgctatcatctttcacacgaagaaaagaaaatctgtgtgcgctgatccaaagcagaactgggtgaaaagggctgtgaacctcctcagcctaagagtcaagaagatgtaaaaaactgatgcttttttgggatggaattggacacagcccaaggaggaaatgatcacagctggggttgaaggcttcacctgcacatcactgcacagacctgatttgtgtcccagtggacttgtccaatggatgaagttgattcatattgcatcatagtgtgtcatatttaagctcacattagagttaagttgtattttatgttatttatagatctgaattttctatgtttagctatttaatgttaatttcccacaatccatgggggcgcttagtggaaggattaatattatgtttaagggaatagtttatatggacctttttgtcaacaataagctattgtaaagatatttaatgttctgtttatttaattgcttcttaaattgatatgattttcttataaaacagaaaagaattataagaatatattgaaaataaaagaattgaaaggtaaaaaaaaaaaaaaaaaaaaaaaaaaaaaaaaaaaaaaaaaaaaaaaaaaa

SEQ ID NO: 21 - DNA, 849 bp, Mus musculus
gagcactcgcagggcactgggtacccagcactgagtacatcaactcctggagctgagaatggcctgcggtggcaagcgtctgctcttccttgctttggcatgggtactgctggctcacctctgcagccaggcagaagcaagcaactacgactgttgcctctcgtacatacagacgcctcttccttccagagctattgtgggtttcacaagacagatggccgatgaagcttgtgacattaatgctatcatctttcacacgaagaaaagaaaatctgtgtgcgctgatccaaagcagaactgggtgaaaagggctgtgaacctcctcagcctaagagtcaagaagatgtaaaaaactgatgcttttttgggatggaattggacacagcccaaggaggaaatgatcacagctggggttgaaggcttcacctgcacatcactgcacagacctgatttgtgtcccagtggacttgtccaatggatgaagttgattcatattgcatcatagtgtgtcatatttaagctcacattagagttaagttgtattttatgttatttatagatctgaattttctatgtttagctatttaatgttaatttcccacaatccatgggggcgcttagtggaaggattaatattatgtttaagggaatagtttatatggacctttttgtcaacaataagctattgtaaagatatttaatgttctgtttatttaattgcttcttaaattgatatgattttcttataaaacagaaaagaattataagaatatattgaaaataaaagaattgaaaggtaaaaaaaaaaaaaaaaaaaaaaaaaaaaaaaaaaaaaaaaaaaaaaaaaaa

SEQ ID NO: 22 - protein, 97 aa, Mus musculus
MACGGKRLLFLALAWVLLAHLCSQAEAASNYDCCLSYIQTPLPSRAIVGFTRQMADEACDINAIIFHTKKRKSVCADPKQNWVKRAVNLLSLRVKKM

SEQ ID NO: 23 - DNA, 1237 bp, Homo sapiens
gctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctaccacacagcagcagttacaaaaaccttccccaggctggacgtggtggctcacgcctgtaatcccagcactttgggaggccaaggtgggtggatcacttgaggtcaggagttcgagaccagcctggccaacatgatgaaaccccatctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgccagggctctgtgaccaggaaggaagtcagcatgcctctagaggcaaggaggggaggaacactgcactcttaagcttccgccgtctcaacccctcacaggagcttactggcaaacatgaaaaatcggcttaccattaaagttctcaatgcaaccataaaaaaaaaa

SEQ ID NO: 24 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 25 - DNA, 579 bp, Mus musculus
cttgcagaggactctgagacagcacatgcatctcccacagcctctgccgcgggtaccatgaagatctctgcagctgccctcaccatcatcctcactgcagccgccctctgcacccccgcacctgcctcaccatatggctcggacaccactccctgctgctttgcctacctctccctcgcgctgcctcgtgcccacgtcaaggagtatttctacaccagcagcaagtgctccaatcttgcagtcgtgtttgtcactcgaaggaaccgccaagtgtgtgccaacccagagaagaagtgggttcaagaatacatcaactatttggagatgagctaggatagagggtttcttgattctgaccctgtatagcttccctgtcattgcttgctctagtcctagccagcttggggatgccactcagtaatcccctactcccactcggtcctgggaaaatgggcatctcagctgctccgaggctctgcacagcaaacccaagaaatcagcatttcattaaaatttcagatgcaaggacaaaaaaaaaaaaaaaaaaaaaaaaaaaaaaaaaaaaaaaaaaaaaaaaaa

SEQ ID NO: 26 - protein, 91 aa, Mus musculus
MKISAAALTIILTAAALCTPAPASPYGSDTTPCCFAYLSLALPRAHVKEYFYTSSKCSNLAVVFVTRRNRQVCANPEKKWVQEYINYLEMS

SEQ ID NO: 27 - DNA, 813 bp, Homo sapiens
agctggtttcagacttcagaaggacacgggcagcagacagtggtcagtcctttcttggctctgctgacactcgagcccacattccgtcacctgctcagaatcatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctgaggggtccagaagcttcgaggcccagcgacctcggtgggcccagtggggaggagcaggagcctgagccttgggaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagatgctcttttaaaaggtaaaaaaaaaaaaaaaaaaa

SEQ ID NO: 28 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 29 - DNA, 804 bp, Mus musculus
gggcatatggcttcagacaccagaaggatacaagcagcagcgagtaccagtcccttttctgttctgctgacaagctcaccctctgtcacctgctcaacatcatgaaggtctccaccactgcccttgctgttcttctctgtaccatgacactctgcaaccaagtcttctcagcgccatatggagctgacaccccgactgcctgctgcttctcctacagccggaagattccacgccaattcatcgttgactattttgaaaccagcagcctttgctcccagccaggtgtcattttcctgactaagagaaaccggcagatctgcgctgactccaaagagacctgggtccaagaatacatcactgacctggaactgaatgcctgagagtcttggaggcagcgaggaaccccccaaacctccatgggtcccgtgtagagcaggggcttgagccccggaacattcctgccacctgcatagctccatctcctataagctgtttgctgccaagtagccacatcgagggactcttcacttgaaattttatttaatttaatcctattggtttaatactatttaattttgtaatttattttattgtcatacttgtatttgtgactatttattctgaaagacttcaggacacgttcctcaacccccatctccctcccagttggtcacactgtttggtgacagctattctaggtagacatgatgacaaagtcatgaactgacaaatgtacaatagatgctttgtttataccagagaagtaataaatatgccctttaacaagtgaaaaaaaaaaaaaaa

SEQ ID NO: 30 - protein, 92 aa, Mus musculus
MKVSTTALAVLLCTMTLCNQVFSAPYGADTPTACCFSYSRKIPRQFIVDYFETSSLCSQPGVIFLTKRNRQICADSKETWVQEYITDLELNA

SEQ ID NO: 31 - DNA, 305 bp, Mus musculus
ctctctggagtctgagtgccctttctaccagccatgaggactctctgctctctgctgctgatatgctgcctccttttctcatataccactccagctgttggaagtttaaaaagtattggatacgaagcagaacttgaccactgccacaccaatggagggtactgtgtcagagccatttgtcctccttctgccaggcgtcctgggagctgtttcccagagaagaacccctgttgcaagtacatgaaatgattagaaggaagcacatggaagtcaagtgacagatgtgtaattgatgtttcaataaa

SEQ ID NO: 32 - protein, 71 aa, Mus musculus
MRTLCSLLLICCLLFSYTTPAVGSLKSIGYEAELDHCHTNGGYCVRAICPPSARRPGSCFPEKNPCCKYMK